(19) United States
(12) Patent Application Publication
(10) Pub. No.: US 2019/0256583 A1
(43) Pub. Date: Aug. 22, 2019

(54) SYSTEMS AND METHODS FOR ALTERING MICROBIOME TO REDUCE DISEASE RISK AND MANIFESTATIONS OF DISEASE

(71) Applicant: SCALED MICROBIOMICS, LLC, Hagerstown, MD (US)

(72) Inventor: Julius G. GOEPP, Hagerstown, MD (US)

(21) Appl. No.: 16/253,392

(22) Filed: Jan. 22, 2019

Related U.S. Application Data
(63) Continuation of application No. PCT/US18/15251, filed on Jan. 25, 2018.
Application No. PCT/US18/15251 is a continuation of application No. PCT/US17/43957, filed on Jul. 26, 2017.
(60) Provisional application No. 62/369,370, filed on Aug. 1, 2016.

(51) Int. Cl. C07K 16/12; A61K 35/741; A61P 1/00

(52) U.S. Cl. CPC C07K 16/12; A61K 35/741; A61K 2035/115; C07K 2317/23; A61P 1/00

(57) ABSTRACT

The present invention relates to the microbiomes of humans and other animals as biomarkers and therapeutic targets for regulation or altering of a microbiome profile associated with states of disease or disease risk, using additive and subtractive therapies aimed at restoring a microbiome profile associated with health, with reduced manifestations of disease, or with reduced disease risk. In particular, the invention provides methods of subtractive therapy using microorganism-specific avian egg yolk-derived immunoglobulins (IgY) in a fashion that titrates, without necessarily eliminating, populations of microorganisms known to be associated with human gut and extraintestinal diseases.

### CROSS REFERENCE TO RELATED APPLICATIONS

This application claims the priority of PCT/US18/15251, filed Jan. 25, 2018, which claims priority to PCT/US17/43957, filed Jul. 26, 2017, which claims priority to 62/369,370 filed Aug. 1, 2016, the entire contents of each of which are hereby incorporated by reference in their respective entireties.

### FIELD OF THE INVENTION

The present invention generally relates to reducing risks and manifestations of disease. More particularly, the present invention relates to microbiomes as biomarkers and therapeutic targets for one or more states of health, disease, and disease risk.

### BACKGROUND OF THE INVENTION

Relationships of Microbiomes With Disease and Disease Risk

A growing number of diseases are associated with alterations and variations in the composition of the communities of microorganisms residing on and within the bodies of humans and other vertebrates. Such communities can be characterized in ecological terms, and variations in ecological factors such as diversity and relative abundance are now recognized as having close associations with specific disorders.

Mechanisms producing the observed associations between microbiomes and states of health remain uncertain, but it is clear that many of such mechanisms involve the metabolic activity of specific taxa of microbes in relation to that of the host, and may relate to energy harvest, to production or modulation of neurotransmitters, and to myriad other interactions between an individual's microbiomes, the individual's own biological and genetic characteristics, and the environment.

Known associations between alterations in

the human intestinal microbiome and disease include but are not limited to cardiometabolic disorders (the metabolic syndrome, obesity, diabetes, cardiovascular risk), cancer risk, neurodegenerative disease risk, behavioral disorders (e.g., autism, attention-deficit hyperactivity disorder (ADHD)), psychiatric disorders including mood disorders, schizophrenia, anxiety, functional gut disorders (e.g., irritable bowel syndrome (IBS), functional diarrhea), other intestinal disorders including celiac disease, non-celiac gluten sensitivity, and small intestinal bacterial overgrowth. While there are thousands of species within the gut microbiome, only a relatively small number of strains, species and higher taxonomic units are presently known to have associations with specific diseases or disease risks. A similar situation applies to the microbiomes found in other body sites, such as the oral cavity, the skin, and the vagina, in which microbiome changes are associated with gingivitis, acne and eczema, and susceptibility to human papillomavirus, respectively.

It has become possible to perform rapid and relatively inexpensive analyses of a subject's microbiome (a “microbiome profile”) from a single specimen or swab, using technology based on identifying microorganism-specific DNA or RNA sequences. Such a microbiome profile may readily be compared with profiles based on large populations of healthy individuals, or with those based on populations of individuals who suffer from a particular disease or disease risk. Such a comparison may suggest that an individual has, or is at risk for, such a disease, based upon the individual's microbiome profile, as has been suggested in the scientific literature. Indicators of disease or disease risk on a microbiome profile may include, but are not limited to, assessments of the absolute or relative abundance of specific microorganism strains, species, genera, or larger taxonomic units, or the diversity of the measured microbiome population.

Living vertebrates have distinctive microbiomes in virtually all body sites that are in contact with the outside world. These include but are not limited to the gastrointestinal (GI) tract (“gut”), the nasopharynx, oropharynx, oral cavity, external car, conjunctiva, skin, vagina, and urethra.

Beneficial Effects of Altering the Composition of a Microbiome

It is known in the art that, in both human and animal models, deliberately altering the composition of a subject's microbiome can alter the subject's risk for, or manifestations of, specific or general disease processes or conditions. Non limiting examples of such intentional alterations include a) replacement therapy, for example, fecal microbial transplant (FMT), in which gut-dwelling microorganisms from fecal matter of a healthy individual are transplanted into the intestinal tract of a diseased subject, b) subtractive therapy, for example, targeted administration of antibiotics to reduce the relative abundance of undesired strains, species, genera, and other taxa of microorganisms, c) additive therapy, for example, administration of live biotherapeutics such as specific probiotic microorganisms or consortia of such microorganisms, to increase the relative abundance of other classes of microorganisms, d) administration of genetically engineered microorganisms capable of producing molecules that themselves alter a given microbiome, and e) administration of prebiotics (i.e. substances that preferentially nourish or otherwise encourage growth of specific classes of microorganisms).

However, each of these approaches has substantial limitations. Fecal microbial transplants are unappealing to many people, and carry at least a theoretical risk of transmission of infectious diseases. Antibiotic administration is limited by potential drug toxicity to the host, unintended destruction of desirable microorganisms, and development of antibiotic resistance both in the subject's intestinal flora and in the community. Live biotherapeutics, probiotic and prebiotic administration are to date limited by difficulty in establishing colonization by desired microorganisms in the desired site, as well as by a lack of precision in knowing which strains should be administered. With the exception of FMT, these approaches also are individually limited by exclusively raising, or exclusively lowering, individual populations of microorganisms, rather than modulating or regulating the measureable microbiome as a whole. Moreover, none of these approaches is capable of rapidly modulating and revising a therapeutic intervention in accordance with changes detected in the microbiome upon treatment.

Passive Immunotherapy with Egg-Derived Immunoglobulins (IgY)

There does exist a class of biological molecules that target, with high specificity, individual strains and species of microorganisms, including but not limited to bacteria, yeasts, viruses, parasites, and fungi. Such molecules are known as immunoglobulins.

In nature, immunoglobulins are found in the blood, the mucous membranes, and the secretions of an animal, and provide partial protection to the animal against a specific, “non-self” entity.

These natural processes can be exploited for production of immunoglobulin molecules useful for therapeutic purposes. By means of immunizing a female vertebrate, it is possible to deliberately stimulate production of one or more immunoglobulins that target one or more microorganisms, or even smaller units, such as peptides, toxins, and the like, that can serve as epitopes to which immunoglobulins will bind. The resulting immunoglobulins can then be harvested from milk, in the case of placental mammals, or from egg yolks, in the case of Avians, specifically, from the eggs of laying hens of domestic chickens, typically of the subspecies Gallus gallus domesticus.

In practice, domestic chickens have proven to be an ideal vertebrate for the production of such immunoglobulins, which, because they are found abundantly in the yolk of the egg, are called “immunoglobulin Y,” or “IgY.” IgY production has now been commercialized, and IgY against virtually any known microorganism, or epitope of a microorganism, can be prepared as desired, at relatively low cost.

To date, however, IgY and other non-human immunoglobulins have been used solely in pursuit of known pathogenic microorganisms or molecules. No literature exists on the use of IgY as a target-specific means of reducing the relative abundance of microorganisms that are not pathogenic per se, but rather represent a threat to a subject simply by their excessive relative abundance, or suppression of healthful diversity.

Live Biotherapeutics

The use of probiotics, living microorganisms with known beneficial functions in the vertebrate digestive tract, is well-established. Published studies suggest that administration of probiotics is effective both in raising the relative abundance of microorganisms in an intestinal microbiome, and in some cases in altering phenotypic expression in the host organism (e.g., altering blood glucose levels). The results of a large number of studies, however, are equivocal as to therapeutic effect, with some studies demonstrating sizeable reductions in symptoms, and others minimal or no change in symptoms following probiotic administration.

One likely explanation for these inconsistent results is that no attempt has yet been made to determine the composition of a subject's microbiome prior to formulation and administration of probiotics. Instead, probiotics have been administered a) entirely without reference to any microbiome profile, or b) only with reference to a general enterotype suggesting that most members of a defined population have specific microorganisms present in low relative abundance. Thus, important real effects of probiotic administration may easily have been obscured by a “one-size-fits-all” approach.

Similarly, no literature exists on dosing or duration of microbiome modulation using probiotics in response to a microbiome profile of a subject or defined population of subjects.

Therefore, there is a need in the art for comprehensive means of utilizing a microbiome as a customized, modifiable biomarker and therapeutic target for identifying and then reducing disease risk or manifestations of disease in an individual over time and in a fashion capable of frequent modulation. This invention addresses that need.

It is essential to note an important distinction between the present invention and publications that have appeared in the medical literature on the use of IgY against pathogenic, disease-causing microorganisms. The microorganisms that contribute, by their high relative abundance in a microbiome, to various states of ill-health, disease, or disease risk, are never frank pathogens. That is, they fail to meet either the first or third of Koch's Postulates, four distinct criteria for establishing a causative relationship between a microorganism and a disease, all four of which must be met to establish such a causative relationship.

Koch's first postulate is that the microorganism must be found in abundance in all subjects suffering from the disease, but should not be found in healthy subjects; in contrast, microorganisms contributing to disease or disease risk in a microbiome are, by definition, part of the normal microbial flora, and are in fact found in a majority of healthy subjects. Furthermore, not all individuals with a microbiome-associated disease necessarily have the same microorganism or microorganisms in excessive relative abundance.

Koch's third postulate is that the microorganism should cause disease when introduced into a healthy subject; in contrast, members of the microbiome that contribute to disease risk when overabundant in a microbiome never directly cause disease when introduced to a healthy subject.

In addition to a lack of prior art on the use of IgY to target non-pathogenic, but disease-associated microorganisms in a microbiome, to date no published research or patents have disclosed a method of formulating microorganism-specific IgY, singly or in a combination of IgY specific to multiple microorganisms, in reference to a known disease- or disease-risk associated microbiome profile, or to a known disease-associated enterotype or microbiome composition. Such a method and formulation would be of interest because individual taxa or operational taxonomic units of microorganisms could be identified and used to specify the component IgY required in the formulation to reduce the relative abundance of the microorganisms of interest, to indirectly raise the relative abundance of other microorganisms that may be suppressed by dominant microorganisms, or to beneficially increase the overall diversity of the microbiome, to the extent that the associated disease is prevented or mitigated, or disease risk is reduced.

The present state of the art also fails to provide a means of modulating any microbiome-directed therapy by adjusting the dose or duration of treatment in response to measured changes in the microbiome. This is important, because it is undesirable to completely eliminate a microorganism member of a microbiome. Also, different subjects may have a given microorganism in increased relative abundance, but at different levels, for example, one subject may have a 20% increase the level seen in healthy populations, while another may have a 40% or greater increase. It is advantageous to be able to provide a proportionate response in such cases.

A further limitation of the present state of the art regarding microbiomes and their impact on human and animal health is the lack of tools enabling specific modifications of microbiomes under experimental conditions. It is currently possible to modify microbiome compositions only in indirect ways, such as administration of antibiotics, living biotherapeutics, or prebiotic formulations, none of which have specific effects at the level of the species. This impediment hampers experimentalists' ability to make specific, controllable changes in the composition of a microbiome and then empirically observe the result. The ability to produce such specific changes in an experimental animal microbiome would permit rapid increases in our understanding of the roles played by individual strains, species or higher taxonomic units in the overall metabolic and metagenomic functions of a microbiome. Such knowledge would also permit increased specificity and utility of a microbiome profile, or determination of microbiome composition, as a clinically-relevant biomarker of health status or disease risk.

### SUMMARY OF THE INVENTION

The following presents a simplified summary in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

As disclosed herein, the present invention includes methods for identifying, preventing, treating, or reducing the risk for a disease associated with characteristics of a microbiome, including, but not limited to, microbiome composition as reported on a microbiome profile, relative abundance, diversity, or ratio of Firmicutes to Bacteroidetes, by favorably altering a microbiome of a subject or members of a defined population of subjects.

In one aspect, the present invention includes a method for preventing, reducing the risk of, or treating, a disease associated with characteristics of a microbiome in at least one subject in need thereof. The method of the invention comprises administering to the at least one subject a therapeutically effective amount of at least one immunoglobulin Y (IgY) specific of at least one microorganism in a microbiome, or of at least one functional microorganism-produced molecule.

In various embodiments of the above aspects, the administered therapeutically effective amount of at least one IgY reduces the relative abundance of the at least one microorganism in the microbiome of the at least one subject.

In another embodiment, the relative abundance of the at least one microorganism is reduced towards its relative abundance on a reference value.

In one embodiment, the at least one microorganism is a non-pathogen selected from the group consisting of bacteria, fungi, yeasts, viruses, prions, parasites, and other life forms comprising a microbiome of the subject.

In another embodiment, the at least one microorganism is a pathogen selected from the group consisting of bacteria, fungi, yeasts, viruses, prions, parasites, and other life forms with pathogenic potential in the subject.

In another embodiment, the at least one microorganism comprises at least one strain, taxon, or operational taxonomic unit (OTU) that is associated with a specific disease or a disease risk when present on a microbiome profile at a relative abundance higher than a reference value.

In another embodiment, the at least one microorganism is associated with a specific disease or a disease risk when present at a higher relative abundance in known populations of subjects who have the disease than in populations of subjects without the disease.

In another embodiment, the at least one IgY targets at least one strain, taxon, or operational taxonomic unit known to be associated with disease or disease risk when present at relative abundance higher than a reference value in the microbiome of the at least one subject.

In another embodiment, the at least one IgY targets at least one pathogenic microorganism-produced molecule known to be associated with disease or disease risk when present in the at least one subject.

In another embodiment, the at least one IgY targets at least one functional microorganism-produced molecule selected from the group including, but not limited to, a lipopolysaccharide (LPS), a flagellin, an adhesion factor, an endotoxin, an exotoxin, a verotoxin, a virulence factor, a bacteriocin, or a peripheral membrane protein.

In a further embodiment, the at least one IgY is administered to the at least one subject on a regular dosing interval for at least once per day and for at least two days.

In yet a further embodiment, the at least one IgY is administered to the at least one subject by a route selected from the group consisting of oral, enteric, intraoral, intraduodenal, intragastric, intravaginal, intra-otic, and topical.

In another embodiment, the at least one subject is monitored for a change in at least one characteristic selected from the group consisting of: a manifestation of a disease or disease risk, a biomarker relevant to a disease or disease risk, a measure of microbiome diversity, and a relative abundance of at least one microorganism as compared with a pre-treatment relative abundance.

In yet another embodiment, when the change is detected in the at least one subject, the treatment with the at least one IgY is modified or terminated.

In another aspect, the invention includes a method for formulating a mixture of IgYs specific of at least one microorganism, or of at least one functional microorganism-produced molecule, in a microbiome of at least one subject in need thereof. The method of the invention comprises identifying the at least one microorganism present at relative abundance higher than a reference value, identifying the at least one microorganism-produced molecule, and customizing the mixture of IgYs to target and reduce the relative abundance of the at least one microorganism or to target the at least one microbiome-produced molecule.

In one embodiment, the relative abundance of the at least one microorganism is determined elevated when compared to a normal microbiome reference level.

In another embodiment, the relative abundance of the at least one microorganism is determined to be similar or elevated when compared to a microbiome profile known to be associated with a specific disease or disease risk.

In another embodiment the at least one functional microorganism-produced molecule is known to be associated with a specific disease or disease risk, or with at least one microorganism found to be in increased relative abundance in the said microbiome of the at least one subject.

In another embodiment, the customized mixture of IgYs is formulated in vivo by immunizing a group of hens against more than one microorganism present in high relative abundance in the least one subject, or against more than one functional microorganism-produced molecule known to be associated with a disease or a disease risk, or with at least one microorganism found to be in increased relative abundance in the microbiome of the at least one subject, wherein all eggs from the group of hens contain the same customized mixture of IgYs.

In a further embodiment, the customized mixture of IgYs is formulated from a stock of IgYs where each IgY is specific of one single microorganism or one single functional microorganism-produced molecule.

In yet a further embodiment, the customized mixture of IgYs reduces the relative abundance of the targeted at least one microorganism towards its relative abundance on a reference value, or towards that reported in studies of populations who lack the disease or disease risk of interest.

In yet another embodiment, the customized mixture of IgYs is packaged in a pharmaceutically acceptable protective agent.

In another aspect, the invention includes a method for increasing the relative abundance of at least one microorganism in a microbiome of at least one subject in need thereof. The method comprises administering to the at least one subject a therapeutically effective amount of at least one live biotherapeutic or probiotic bacterial species, wherein the need of the at least one subject is determined by detecting a relative abundance of the at least one microorganism lower than a reference value in the microbiome of the at least one subject.

In one embodiment, the at least one probiotic bacterial species is a non-pathogen or a non-pathogen probiotic species or live biotherapeutic strain known to increase the relative abundance of microorganisms found in the microbiome of the at least one subject.

In another embodiment, following administration of the therapeutically effective amount of at least one probiotic microorganism, the relative abundance of the at least one microorganism is increased towards its relative abundance on a reference value, or towards that reported in studies of populations who lack the disease or disease risk of interest.

In another embodiment, the administered at least one live biotherapeutic or probiotic species comprise at least one strain, taxon, or operational taxonomic unit that is associated with specific diseases or disease risks when present in low relative abundance in a microbiome when compared with a reference value, or that is present in similar or reduced abundance when compared to a microbiome profile known to be associated with a specific disease or disease risk.

In another embodiment, the at least one probiotic species is produced by specific culture methods favoring growth of the desired probiotic microorganisms.

In another embodiment, the therapeutically effective amount of the at least one probiotic bacterial species contains quantities of at least one microorganism targeted to raise the relative abundance of at least one strain, taxon, or operational taxonomic unit known to be associated with disease or disease risk when present at low relative abundance in a microbiome.

In a further embodiment, the therapeutically effective mixture of the at least one probiotic bacterial species is administered to the at least one subject on a regular dosing interval for at least once per day and for at least 7 days.

In yet a further embodiment, the therapeutically effective mixture of the at least one probiotic bacterial species is administered to the at least one subject by a route selected from the group consisting of oral, enteric, intraoral, intraduodenal, intragastric, intravaginal, otic, and topical.

In another embodiment, the at least one subject is monitored for a change in at least one characteristic selected from the group consisting of: a manifestation of a disease or disease risk, a biomarker relevant to a disease or disease risk, a measure of microbiome diversity, and a relative abundance of at least one microorganism as compared with the pre-treatment values.

In yet another embodiment, when the change is detected in the at least one subject, the treatment with the therapeutically effective mixture of the at least one probiotic bacterial species is modified or terminated.

In another aspect, the invention includes a method for formulating a therapeutically effective mixture of probiotics specific of at least one microorganism in a microbiome of at least one subject in need thereof. The method of the invention comprises identifying at least one microorganism present in a relative abundance lower than a reference value in a microbiome profile of the at least one subject, or similar to or lower than that reported in studies of subjects with the disease of interest, wherein the mixture of probiotics is customized to target and increase the relative abundance of the at least one microorganism.

In one embodiment, the relative abundance of the at least one microorganism in the at least one subject's microbiome is determined to be decreased when compared to a normal microbiome reference.

In another embodiment, the relative abundance of the at least one microorganism in the at least one subject's microbiome profile is determined to be similar or decreased when compared to a microbiome profile known to be associated with a specific disease or disease risk.

In a further embodiment, the customized mixture of probiotics is formulated from stocks of living probiotic microorganisms.

In yet a further embodiment, the customized mixture of probiotics is formulated with doses of individual probiotic microorganisms calculated to increase the relative abundance of each targeted microorganism towards its relative abundance on a reference value.

In yet another embodiment, the customized mixture of probiotics is packaged with a pharmaceutically acceptable protective agent.

In some embodiments, the microbiome is selected from the group consisting of intestinal, gastric, oral, oropharyngeal, otic, nasal, nasopharyngeal, skin, axillary, vaginal, and conjunctival microbiomes.

In some embodiments, the at least one subject is a human.

In some embodiments, the at least one subject is an animal selected from the group consisting of: a primate, a rodent, a feline, a canine, a poultry, an avian and other domesticated animals.

In other embodiments, the at least one subject is a member of a defined population having in common a disease or risk for a disease.

In other embodiments, the microbiome profile is representative of a defined population having in common a disease or risk for a disease.

In yet other embodiments, the relative abundance of the at least one microorganism is determined to be elevated when compared to a normal reference of a defined healthy population.

In still another embodiment, the relative abundance of the at least one microorganism is determined to be similar to or elevated when compared to a reference level of a defined population having in common a disease or a disease risk.

In another embodiment, the relative abundance of the at least one microorganism is determined to be reduced when compared to a reference value of a defined healthy population.

In further embodiments, the relative abundance of the at least one microorganism is determined to be similar or reduced to a reference value of a defined population having in common a disease or risk for a disease.

In yet further embodiments, the at least one subject has or is at risk for or having at least one disease selected from the group consisting of irritable bowel syndrome, small intestinal bacterial overgrowth, inflammatory bowel disease, functional bowel disorders, celiac disease, non-celiac gluten sensitivity, migraine, cardiovascular disease, type 1 or type 2 diabetes, obesity, the metabolic syndrome, osteoporosis, autoimmune disease, osteoporosis, behavioral disorder, malignancy, cutaneous disorders (e.g. acne, eczema), gingivitis, periodontitis, vaginal papillomavirus infections, and psychiatric disorder.

In another aspect, the invention includes a method for research into microbiome composition and dynamics in order to develop improved knowledge of the effect of any individual strain, species, genus, or higher taxonomic unit in a microbiome.

In one embodiment, at least one IgY molecule specific of individual strains, species, genera, or higher taxonomic units of microorganisms in a microbiome is prepared by immunization of laying hens with antigens derived from said microorganisms. The at least one organism-specific IgY is then administered to laboratory animals or in vitro models of functioning microbiomes, and the resulting alterations in microbiome composition and function are studied.

In another embodiment, the at least one organism-specific IgY is formulated into feed pellets or other forms suitable for dietary administration to laboratory animals.

In another embodiment, the at least one organism-specific IgY is formulated in a non-interacting vehicle suitable for administration by intragastric, intraduodenal, or other enteral administration separate from the research animal's diet.

In another embodiment, the at least one organism-specific IgY is administered directly into a laboratory model microbiome.

In still a further embodiment, the at least one organism-specific IgY is formulated for administration topically, intraorally, or intravaginally, to study selected disruptions of microbiomes on skin, in the oral cavity, or in the vagina.

In yet a further embodiment, the specific IgY molecules are developed to target functional microorganism-produced molecules, at least one of which is incorporated into feed or prepared for direct administration as described above.

### BRIEF DESCRIPTION OF THE DRAWINGS

For a more complete understanding of the nature and desired objects of the present invention, reference is made to the following detailed description taken in conjunction with the accompanying drawing figures wherein like reference characters denote corresponding parts throughout the several views.

FIG. 1 provides an overview, demonstrating initial fecal testing for microbiome composition, use of that data for formulation of a mixture to contain microorganism-specific IgY immunoglobulins against those microorganisms found in excessive relative abundance, and live microorganisms selected to increase the relative abundance of those found in diminished relative abundance, followed by monitoring, re-testing, and reformulation of the mixture as required. It is understood that these and the following steps may be applied equally to at least one single subject to achieve a personalized result, or to a group of subjects with or at risk for a microbiome-related disease or condition (for illustrative example, irritable bowel syndrome). In the former case the microbiome composition is determined by an individual specimen from the at least one single subject, while in the latter the microbiome composition in estimated from studies of groups of subjects with or at risk for the microbiome-related disease or condition.

FIG. 2 provides a more detailed diagram of the initial steps of obtaining a fecal sample from a subject or a group of subjects having in common a disease or a disease risk, and performing analyses to detect and quantify the intestinal microbiome composition, production of a related report, and transmission of said report to a formulating facility. Shown also is the analysis of fecal specimen for selected biomarkers indicative of state(s) of disease, disease risk, or health. In the case of a group of subjects, this process is understood to take place in the form of study(ies) capable of discerning significant differences in microbiome composition between subjects with or at risk for the disease or condition and those without it. Such study may occur as a part of the formulation of the therapeutically effective mixture, or may be previously published data, in which case data for formulation are derived from said previous study.

FIG. 3 illustrates a schematic of procedures, for producing microorganism-specific IgY in bulk, in response to an individual microbiome profile or one derived from a group of subjects having or at risk for a microbiome-related disease or condition, at an agricultural facility for laying hens, pooling yolks and extracting specific IgY, affinity-purifying IgY as required, accumulating stocks of individual microorganism-specific IgY in gram, kilogram, or larger quantities, and shipping IgY to formulating facility.

FIG. 4 illustrates a schematic of procedures, for raising bulk quantities of specific probiotic microorganisms at a microbiological facility, then packaging them using a means that preserves their viability, for shipment to a formulating facility.

FIG. 5 illustrates the steps that will be taken at the formulating facility for preparation of personalized combinations of microorganism-specific IgYs and live probiotic microorganisms.

FIGS. 6A, 6B, 6C and 6D illustrate the target-specificity and cross-reactivity of each of the 14 IgYs on ELISA testing in Example 1. Each target-specific IgY is tested against all bacteria in the group. Each target-specific IgY binds best to its target bacteria, and shows varying degrees of cross-reactivity with other bacteria. With the exceptions of E. coli, K. pneumoniae, and E. faecalis, control IgY shows little reactivity with targeted species.

FIGS. 7A, 7B, 7C and 7D provide a series of graphs illustrating the growth rate of each targeted organism in the presence of no treatment (Untreated), control IgY (Control), and target-specific IgY (Exp) in Example 1.

### DETAILED DESCRIPTION

### Definitions:

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rigger et al. (eds.). Springer Verlag (1991); and Hale & Marham, The Harper Collins Dictionary of Biology (1991). As used herein, the following terms have the meanings ascribed to them below, unless specified otherwise.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below.

As used herein, the term “about” will be understood by persons of ordinary skill in the art and will vary to some extent on the context in which it is used. As used herein when referring to a measurable value such as an amount, a concentration, a temporal duration, and the like, the term “about” is meant to encompass variations of ±20% or ±10%, more preferably ±5%, even more preferably 1%, and still more preferably ±0.1% from the specified value, as such variations are appropriate to perform the disclosed methods.

As used herein, “abnormal/disease reference” means a range of values associated with elevated risk established by studies of populations determined to have, or be at risk for, one or more specific diseases or disability states, a range of values associated with elevated risk established by publically-available microbiome libraries of microbiome profiles of populations determined to have, or be at risk for, one or more specific diseases or disability states, a range of values associated with elevated risk established by private individuals, governmental, academic, nonprofit, or corporate entities that develop or maintain such libraries of microbiome profiles of population determined to have, or be at risk for, a specific disease or disability state.

As used herein, “absolute abundance” means the measured amount, (for non-limiting example, in colony-forming units, or CFU) of a given taxonomic unit in a microbiome.

As used herein, “additive therapy” means the addition of specific microorganisms to a microbiome found to have those microorganisms present at reduced relative abundance. The most common example of additive therapy is the use of probiotics.

By “ameliorate” is meant decrease, suppress, attenuate, diminish, arrest, or stabilize the development or progression of a disease.

As used herein, “animal” refers to living multi-cellular vertebrate organisms, a category that includes, for example, mammals and birds. The term “mammal” includes both human and non-human mammals.

As used herein, “Bacteroidetes” refers to the bacterial phylum Bacteroidetes, or to any microorganism classified as a member of that phylum.

As used herein, “immunoglobulin” refers to a polypeptide ligand comprising at least a light chain or heavy chain immunoglobulin variable region which specifically binds an epitope of a protein or a fragment of a protein. Immunoglobulins can include a heavy chain and a light chain, each of which has a variable region, termed the variable heavy (VH) region and the variable light (VL) region. Together, the VH region and the VL region are responsible for binding the antigen recognized by the immunoglobulin. This includes intact immunoglobulins and the variants and portions of them well known in the art, such as Fab′ fragments, F(ab)′2 fragments, single chain F proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes recombinant forms such as chimeric immunoglobulins (for example, humanized murine immunoglobulins), heteroconjugate immunoglobulins (such as, bispecific immunoglobulins), and immunoglobulins produced by genetically-modified bacteria or yeast under defined conditions. See also, Pierce Catalog and Handbook, 1994-1995 (Pierce Chemical Co., Rockford, Ill.); Kuby, Immunology, 3rd Ed., W.H. Freeman & Co., New York, 1997.

As used herein, “biomarker” refers to any measurable analyte used in the diagnosis or assessment of a disease, disease process, or disease risk; nonlimiting examples include relative abundance and diversity as characteristics of a microbiome, fecal calprotectin as an indicator of intestinal inflammation, or serum cholesterol as an indicator of cardiovascular disease risk.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

As used herein, “customized” means developed in response to one or more measured characteristic of an individual or population, said characteristic being relevant to a microbiome profile. For example, a mixture of IgYs is “customized” to the needs of a subject or group of subjects when it is formulated by reference to a microbiome profile of said subject or group of subjects. When “customized” is used in reference to an individual subject, or a formulation for use in a specific individual subject, the term includes the sense of “personalized.”

As used herein, “defined population” means a distinct population of individuals having in common at least one common characteristic relevant to disease or disease risk. Nonlimiting examples of defined populations include all those living in a defined geographical or climatic area, those relying on a common water source, those with serum cholesterol levels within a specified range, those with defining symptoms of a disease (e.g., irritable bowel syndrome), those at elevated risk for cancer, those at elevated risk for neurodegenerative diseases, and those with psychiatric, behavioral, or mood disorders.

By “disease” is meant any condition or disorder that damages or interferes with the normal function of a cell, tissue, or organ, system or entire organism.

As used herein, “diversity” means a measure of the number of distinct genera, species, and other taxa or operational taxonomic units present within a defined microbiome from a body site. Common indices of diversity include, but are not limited to, the Shannon index and the Simpson index.

The terms “effective amount” or “therapeutically effective amount” are used interchangeably herein, and refer to an amount of a compound, formulation, material, or composition, as described herein effective to achieve a particular biological result. Such results may include, but are not limited to, the treatment of a disease or condition as determined by any means suitable in the art.

As used herein, “epitope” means a molecular structure found on a microorganism, or represented by a functional microorganism-produced molecule, that is recognized by the immune system and leads to the production of specific immunoglobulins directed against the epitope.

As used herein, “Firmicutes” or “Firmicute” refers to the bacterial phylum Firmicutes, or to any microorganism classified as a member of that phylum.

As used herein, “normal reference” and “healthy reference” mean a reference range or typical microbiome composition established by studies of microbiome compositions of populations determined to be in states of good health, a reference range established by publically-available microbiome libraries of microbiome profiles of populations in states of good health, and a reference range established by private individuals, governmental, academic, nonprofit, or corporate entities that develop or maintain such libraries of microbiome profiles of populations in states of good health.

As used herein, “immunoglobulin Y” (“IgY”) is a type of immunoglobulin which is the major immunoglobulin in bird, reptile, and lungfish blood. It is also found in high concentrations in chicken egg yolk. As with the other immunoglobulins, IgY is a class of proteins which are formed by the immune system in reaction to certain foreign substances, and specifically recognize them. IgY is composed of two light and two heavy chains. Structurally, these two types of immunoglobulin differ primarily in the heavy chains, which in IgY have a molecular mass of about 65,100 atomic mass units (amu). The light chains in IgY have a molar mass of about 18,700 amu. The molar mass of IgY thus amounts to about 167,000 amu.

As used herein, “IgY specific of one or more microorganisms” means IgY molecules produced by immunizing a hen with one or more specific antigens derived from one or more specific microorganisms, such that the IgY produced is capable of binding to said antigens when encountered by the IgY molecule.

“Immunoassay” refers to a biochemical test that measures the presence or concentration of a substance in a sample, such as a biological sample, using the reaction of an immunoglobulin to its cognate antigen, for example the specific binding of an immunoglobulin to a protein. Both the presence of antigen or the amount of antigen present can be measured. For measuring proteins, for each the antigen and the presence and amount (abundance) of the protein can be determined or measured. Measuring the quantity of antigen can be achieved by a variety of methods. One of the most common is to label either the antigen or immunoglobulin with a detectable label.

An “individual”, “patient” or “subject”, as these terms are used interchangeably herein, includes a member of any animal species including, but are not limited to, birds, humans and other primates, and other mammals including commercially relevant mammals such as cattle, pigs, horses, sheep, cats, and dogs. Preferably, the subject is a human.

As used herein, “In vivo” refers to processes that take place within a living organism, herein most commonly, a laying hen.

As used herein, “manifestation of a disease” means any sign, symptom, diagnostic test result, severity score, quality-of-life measure, or measure of disability that is associated with a specific disease, disease process, or disease risk.

As used herein, “metagenomic analysis” means an analysis, by culture-independent and sequencing-based studies, of the collective set of genomes of mixed microorganism communities (metagenomes), with the aim of exploring their compositional and functional characteristics.

As used herein, “microbiome” or “microbiota” refers to the ecological community of commensal, symbiotic and pathogenic microorganisms that are normally found dwelling in specific sites of an animal or human body. The term is used to emphasize the importance of microorganisms inhabiting the human body in health and disease. By the original definitions the terms “microbiome” and “microbiota” are largely synonymous, and are used synonymously herein. Nonlimiting examples of individual microbiomes include the intestinal (“gut”) microbiome, the oropharyngeal microbiome, the skin microbiome, and the vaginal microbiome.

As used herein, “microbiome composition” means the kind, amount, and diversity of detectable microorganisms in a microbiome. “Microbiome composition” in this sense is the biomarker that indicates the state of an individual microbiome or that of microbiomes typical to a group of subjects with or at risk for a disease or condition.

As used herein, a “microbiome profile” is a quantitative report revealing the kind and amount of selected microorganisms (“microbiome composition”) present in a sample of a microbiome. In this sense the “microbiome profile” is the laboratory report that provides in formation about the biomarker, “microbiome composition.” Components of the report include but are not limited to diversity, relative abundance of specific strains, species, genera, and other taxonomic units, and the Firmicutes/Bacteroidetes (F/B) ratio. A microbiome profile may be generated by at least one of a multiplicity of techniques known to those in the art, for example, multiplex real-time polymerase chain reaction (PCR), pyrosequencing, whole genome sequencing, shotgun analysis, and others.

As used herein, “microorganism” refers to any microscopic life form capable of self-replication. Nonlimiting examples include bacteria, viruses, protozoans, prions, fungi, and yeasts.

As used herein, “naturally-occurring pathogenic molecules” refers to molecules that occur in nature in foods or in the environment, and that are known to play an active role in disease production. A common example of a naturally-occurring pathogenic molecule is the wheat protein, gliadin, which becomes pathogenic if it passes from the intestinal lumen into the bloodstream, where it can trigger an autoimmune reaction in subjects having the necessary genetic pre-load.

As used herein, “non-pathogen” refers to a microorganism that does not fulfill Koch's Postulates establishing a causative relationship between a microorganism and a disease, but rather that is a natural or normal member of a microbiome.

By “defined microbial consortia” is meant a purified and/or isolated population of known microbes.

By “undesirable gut microbiome” is meant a community of microbes comprising a pathogen or having a biological activity associated with a pathogenic process.

By “normal gut flora” is meant a population of microbes that is substantially similar to the population of microbes present in the gut of a healthy control subject or a group of subjects determined to be in good health and free of the disease of interest.

As used herein, a “normal microbiome reference level” means the approximate relative abundance of at least one microorganism comprising a microbiome of a subject in good health, or the range of relative abundance of said at least one microorganism comprising a representative microbiome of a group or population of subjects determined to be in good health, or to have been demonstrated not to have or be at risk for the microbiome-related disease or condition of immediate interest.

As used herein, “nucleotide” means the organic molecules that serve as subunits of nucleic acids such as DNA and RNA. The terms “nucleotide sequence,” “DNA sequence,” and “RNA sequence” refer to covalently-bonded linear sequences of nucleotides, DNA, or RNA that can be detected using automated biochemical methods know to those skilled in the art. Living organisms contain many unique nucleotide sequences, and the detection of such sequences forms the basis for identification of specific organisms present.

As used herein, “operational taxonomic unit” means a group of individual microorganisms closely related on the basis of close similarity of DNA or RNA sequences.

As used herein, “taxon” and “taxa” refer to the singular and plural, respectively, of one or more groups of one or more populations of an organism or organisms seen by taxonomists to form a unit in the hierarchy of the organization of life (taxonomy). Common examples of taxa include, but are not limited to, phylum (plural phyla), class, order, family, genus, species, subspecies and strains.

As used herein, “pathogen” means a microorganism that does fulfill Koch's Postulates establishing a causative relationship between a microorganism and a disease, that is, the pathogen always produces the infectious disease, and in the absence of the pathogen, the disease never occurs.

As used herein, “functional microorganism-produced molecule” means a molecule, produced by at least one microorganism taxon, that produces biological effects in a host of a microbiome. Such molecules may facilitate communications between microorganisms in a microbiome, between microorganisms and the host, or may be used by one taxon of microorganisms to suppress growth of other taxa (e.g., bacteriocins). Functional microorganism-produced molecules may also directly produce disease (e.g., bacterial exotoxins or endotoxins), or may contribute to disease or disease risk by means of their interactions with host tissues (e.g., virulence factors).

As used herein, “pharmaceutically acceptable protective agent” means a coating, encapsulation, covalent bond, physical structure, or other means of protecting the therapeutic mixture of IgY or probiotics against degradation in the stomach or upper gastrointestinal tract. Examples of such protective agents include, but are not limited to, acid-resistant capsules, liposomes, nanospheres, and other methodologies known to those practiced in the art.

As used herein, “prebiotic” means a non-living organic compound known to foster the growth or colonization of a probiotic microorganism.

As used herein, “probiotic” or “live biotherapeutic” means ingested microorganisms associated with benefits for humans and animals. The terms are used interchangeably herein. A probiotic formulation may contain multiple individual organisms from a single species or other taxonomic unit, or may be a mixture of multiple taxonomic units.

As used herein, “reference value” for a microbiome profile reporting a microbiome composition means at least one of the following: the relative abundance of at least one microorganism in a microbiome profile generally recognized as representative of a group of subjects in good general health, the relative abundance of at least one microorganism in a microbiome profile generally recognized as representative of a group of subjects having in common the absence of a specific disease or disease risk, or the relative abundance of at least one microorganism in a microbiome profile generally recognized as representative of a group of subjects having in common a disease or a disease risk. It is understood that the expression “higher than a reference value” means that the relative abundance of at least one microorganism on an individual subject's microbiome profile, or the microbiome profile of a group of subjects, is greater than that of the reference value to a degree known to be associated with a disease or a disease risk. It is further understood that the expression “lower than a reference value” means that the relative abundance of at least one microorganism on an individual subject's microbiome profile, or the microbiome profile of a group of subjects, is lower than that of the reference value to a degree known to be associated with a disease or a disease risk.

As used herein, “relative abundance” refers to the proportional contribution of a taxonomic unit to the overall composition of a microbiome. For example, in a theoretical, simplified microbiome containing five total taxonomic units of organisms, all found in identical proportions, each taxonomic unit would have a relative abundance of 20%, while in another, two taxonomic units might be present at 35% each, and the remaining three would then be present at 10% each.

As used herein, “replacement therapy” refers to a complete, or near-complete, replacement of an entire microbiome with microorganisms from another source. The most relevant and common example of replacement therapy for an intestinal microbiome is a fecal microbial transplant (FMT), in which microorganisms from fecal matter of a healthy subject are transferred into the colon of a subject in need of modulation of the intestinal microbiome, with the result that the microorganisms establish and maintain a new microbiome in the intestine of the recipient.

As used herein, “subtractive therapy” means the deliberate reduction in absolute or relative abundance of at least one microorganism in a microbiome, with the intent of modulating a microbiome to more closely resemble one associated with health. Examples of subtractive therapy include the use of targeted antibiotics, and the use of the still more highly-targeted IgY.

As used herein, “therapeutic agent” refers to a substance that demonstrates some therapeutic effect by restoring or maintaining health, such as by alleviating the signs, symptoms, or abnormal diagnostic test results associated with a disease or physiological disorder, or delaying (including preventing) progression or onset of a disease. In some instances, the therapeutic agent is a chemical, biological, or pharmaceutical agent, or a prodrug. A therapeutic agent may be an agent which prevents or inhibits one or more signs or symptoms or laboratory findings associated with disease or disease risk.

A “therapeutically effective amount” or “effective amount” or “therapeutically effective dose” is that amount or dose sufficient to inhibit or prevent onset or advancement, to treat outward symptoms, or to cause regression, of a disease. The therapeutically effective amount or dose also can be considered as that amount or dose capable of relieving symptoms caused by the disease. Thus, a therapeutically effective amount or dose of an anti-fungal agent is that amount or dose sufficient to achieve a stated therapeutic effect. The therapeutically effective amount may vary depending the subject and disease condition being treated, e.g., the weight and age of the subject, the severity of the disease condition, the manner of administration and the like, which can readily be determined by one of ordinary skill in the art.

As used herein, “topical administration” means application or administration of a therapeutically effective amount of a substance (here, IgY or probiotic microorganisms) to the skin or mucous membrane surfaces of a living vertebrate.

As used herein, the terms “treatment” and “treating” refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description:

There exists a long-felt need to modify the composition of a microbiome towards one associated with health, or at least reduced risk for or manifestations of a disease. Such a modification ideally includes means for reducing the relative abundance of some microorganisms, increasing the relative abundance of other microorganisms, or increasing the overall diversity of microorganisms within a microbiome.

To date, no comprehensive and specifically targeted means for such alteration exists, hampering efforts to respond clinically to known associations between microbiome disruptions (dysbiosis) and disease manifestations or disease risk.

This invention discloses methods for reducing the risk of, or treating, a disease associated with characteristics of a microbiome in at least one subject in need thereof, the method comprising administering to the at least one subject a therapeutically effective amount of at least one IgY specific of at least one microorganism in a microbiome or at least one functional microorganism-produced molecule.

In one aspect of this invention, a mixture of IgY is devised, such that the IgY immunoglobulins target microorganisms in excessively high relative abundance in a microbiome, and work to reduce their relative abundance. In a further aspect, the IgY mixture contains IgY immunoglobulins that target specific molecules known to be produced by microorganisms in a microbiome, and work to reduce concentrations of such microorganism-produced molecules.

Additionally, in some embodiments, selected probiotic microorganisms in low relative abundance in a microbiome are added to the IgY mixture and work to increase their abundance. By reducing levels of overabundant microorganisms or microorganism-produced molecules, and by raising levels of under-abundant microorganisms, a disordered microbiome associated with disease or disease risk can be manipulated towards closer resemblance to those of people in states of health.

In other embodiments, the methods disclosed in this invention are used to generate mixtures of IgY or probiotic microorganisms that are customized to an individual subject, by the expedient of first obtaining a baseline microbiome profile from the subject, and using the results to guide formulation of the mixture. In some embodiments, the processes disclosed in this invention are also used to generate mixtures of IgY or probiotic microorganisms that are customized to a defined or definable population whose members have in common a disease or an increased risk for a disease, and with which a typical pattern of disordered microbiome is associated.

In still other embodiments, the methods disclosed in this invention are used in test kits, strips, ELISA, latex-agglutination, or other antigen-detection systems to specifically identify key microorganisms known to be associated with disease or disease risk when found in excessively high or low abundance in a microorganism. In some embodiments, such systems are used as point-of-care testing in healthcare facilities, while in other embodiments such systems are used in non-professional settings to provide indications of the overall health of a microbiome based on a specimen selected from one of fecal, oral, gingival, nasal, vaginal, skin, scalp or other body sites.

This invention solves several problems that are associated with the current state of the art of modifying a microbiome associated with disease or disease risk towards one associated with health or reduced disease risk.

First, the use of IgY permits targeting of individual strains or species of microorganisms with much greater specificity than can be accomplished with antibiotic use, which inevitably destroys microorganisms other than those targeted for intervention. That degree of specificity is further enhanced in this invention because the mixture of IgY or probiotics is formulated in response to a known disordered microbiome profile of a subject or a population, rather than applying a “one-size-fits-all” approach, as is the case when antibiotics or unselected probiotics are used.

Further advantages of the invention include the ability to continuously reformulate the IgY or probiotic mixture in response to microbiome profiles obtained after a period of treatment, to permit increased or decreased dosing of the specific IgY or probiotics in the mixture, and the lack of development of resistance to IgY, as opposed to the high risk of microorganisms developing resistance to antibiotics, particularly if repeatedly or continuously administered.

The present invention provides a novel approach to “subtractive therapics” aimed at the microbiomes of humans and other animals, when such microbiomes demonstrate a pattern associated with disease or increased disease risk. In such microbiomes, it is desirable to alter the relative abundance of various species or other taxa known to be associated with various states of disease, disease risk, and health. It is also desirable in general to raise the overall diversity of a microbiome, an effect that may be achieved by selective reduction in the numbers of dominant microorganisms in a microbiome.

The present invention also provides a novel approach to research on microbiomes in living animals or in model microbiome systems, by allowing researchers to selectively and specifically alter the abundance of individual strains, species, or other taxa and observing the impact on microbiome composition, function, and host phenotype. This is a desirable new capability because current science does not permit such selective modifications, limiting the study of the contribution of any individual microorganism or groups of microorganisms to host biological function.

A related innovation of the present invention is the ability for researchers to rapidly determine the impact on a microbiome's composition by use of target-specific IgY-based detection systems capable of producing a quantitative indication of the abundance of microorganisms associated with disease or a disease risk, prior to, during, or following experimentation on a microbiome by selective use of target-specific IgY intended to alter the abundance of targeted microorganisms.

In one aspect, the invention provides a method of reducing the relative and absolute abundance of microorganisms that, when found to be in high relative abundance, are associated with known states of disease or disease risk, that means being the administration of avian immunoglobulins (IgY) derived from the egg yolks of specially-immunized hens. The choice of which microorganism-specific IgY(s) is to be administered is determined by sampling a microbiome (here, in non-limiting example, a gastrointestinal microbiome) of a subject or of a population of subjects having in common a disease or risk for a disease, in a fashion that produces a quantitative report of the absolute or relative abundance of selected microorganisms whose relative abundance has been associated with disease or disease risk. Similarly, the choice of microorganism-specific IgY molecules for inclusion in a formulation may be driven by consultation with published studies of predominant microbiome compositions in specific diseases or states of increased disease risk.

In a similar aspect, the invention includes a method of administering an IgY specific of a functional microorganism-produced molecule known to be associated with disease or disease risk. Non-limiting examples of a functional molecule are a lipopolysaccharide, a flagellin, an adhesion factor, an endotoxin, an exotoxin, a verotoxin, a virulence factor, a bacteriocin, or a peripheral membrane protein. The decision to include such IgY(s) into a formulation can be driven by a microbiome profile demonstrating presence of microorganisms known to produce such molecules, by consultation with published literature, or by detection of such molecules in biological samples from the microbiome site.

As a result, the administering of the customized IgY mixture is expected to result in selective reduction (i.e. subtractive therapy) of microorganisms or of functional microorganism-produced molecules, and therefore in a change of the composition of the subject's or population's microbiome, to more closely resemble a microbiome associated with health. A variety of methods known in the art for administering the IgY composition of the invention are contemplated by the present invention. The composition described herein may be administered to a patient transafterially, subcutaneously, intradermally, intratumorally, intranodally, intramedullary, intramuscularly, by intravenous (i.v.) injection, intraperitoneally, topically, intraorally, intravaginally, or by ingestion. In other instances, the IgY(s) of the invention are injected directly into a site of inflammation in the subject, a local disease site in the subject, a lymph node, an organ, a tumor, and the like. In other embodiments, this invention is useful for improving the characteristics of microbiomes from sites other than the gastrointestinal tract, and to non-human animals.

In the following description, examples and figures, the methods of the invention should not be construed as being limited solely to the microbiome of an individual subject but rather applicable as well to the microbiome of a population. It is understood that the processes described apply both to the modification of an individual subject's microbiome, and to that of a microbiome representative of a population of subjects having in common a disease or disease risk influenced by microbiome composition. In the case of populations of subjects, the microbiome profile is one representative of the population of interest, derived from population-level studies. For instance, if the population of interest represents patients with irritable bowel syndrome (IBS), then the microbiome profile is understood to mean an aggregate microbiome profile representative of a known population of IBS patients.

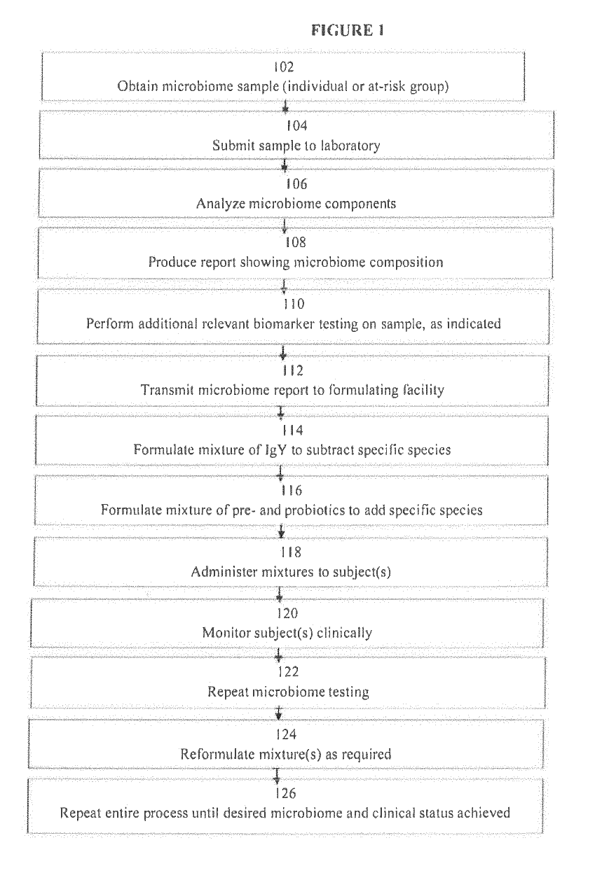

FIG. 1 provides an overview of one embodiment, demonstrating initial fecal testing for microbiome characterization, use of that data for formulation of a mixture to contain microorganism-specific IgY immunoglobulins against those microorganisms found in excessive relative abundance, and/or live microorganisms selected to increase the relative abundance of those found in diminished relative abundance, followed by monitoring, re-testing, and reformulation of the customized mixture as required.

In at least one embodiment the process of the present invention is used in conjunction with an individual subject with, or at risk for, disease or disease risk influenced by microbiome composition. In at least one other embodiment, the process of the present invention is used in conjunction with a population of subjects having in common a disease or disease risk influenced by microbiome composition.

As illustrated by FIG. 1, a microbiome sample is obtained (102), the sample is submitted to a laboratory (104), a metagenomic analysis of microbiome composition is performed (106), a related report is produced (108) and ancillary biomarker testing is performed if indicated (110). In this embodiment, the microbiome report is transmitted to a formulating facility (112) where multiple IgYs against target microorganisms, and multiple probiotic microorganisms, are stored in bulk. A customized mixture of IgYs is next formulated (114) aimed at subtracting the species that are in overabundance, and a similar mixture of probiotic microorganisms is prepared (116) aimed at increasing species that are under-abundant. When functional microorganism-produced molecules are known or suspected to be present and contributing to a disease process or an increased risk for a disease, the IgY mixture is formulated to include IgY immunoglobulins specific of such molecules. The mixtures are then administered to the Subject (118), and the Subject is monitored for clinical outcomes (120) (e.g., improvements in manifestations of a disease, or measurably improved biomarkers). Steps (102) through (110) are repeated (122), and the IgY and probiotic mixtures are reformulated in response to the second microbiome report (124). The entire process is repeated (126) through as many cycles as required to achieve the desired clinical status and microbiome profile.

All of the steps described in FIG. 1 may also be performed for the benefit of a group of individuals having in common a disease or disease risk associated with dysbiosis, in which case the microbiome profile is one representative of a large number of members of such a group, and the IgY or probiotic mixture is formulated in response to the representative group microbiome profile. The resulting mixture is then made available for administration to members of the group having in common a disease or disease risk, and members of the group are individually monitored for clinical outcomes. The repetition of the cycle, including alterations of the IgY and probiotic mixture, and the repeated microbiome profile determinations, are understood to be optional parts of the overall process. Similarly, the process may be divided into separate subtractive and additive parts, either of which may be administered alone or in combination with the other, such that a formulation may be developed to contain only microorganism-specific IgY, only specific live biotherapeutics or probiotics, or both kinds of components.

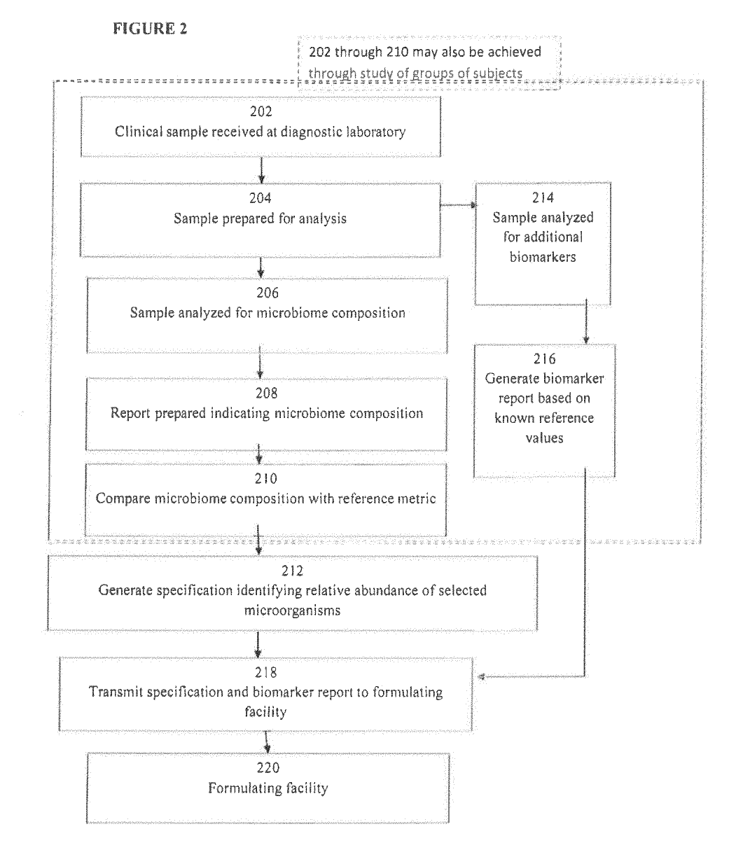

FIG. 2 provides a more detailed diagram of the initial steps of obtaining a fecal sample from a subject and performing analyses to detect and quantify the subject's microbiome profile, production of a related report, and transmission of said report to a formulating facility. It is understood here that the initial microbiome profile may be that representative of a group of subjects having in common a disease or a disease risk. Shown also is the optional analysis of specimen for selected biomarkers indicative of state(s) of disease, disease risk, or health.

As illustrated by FIG. 2, a microbiome sample is received by the laboratory (202), where it undergoes specimen preparation (204). The prepared specimen is then subjected to metagenomic analysis (206), and a report (208) is generated, indicating the diversity, absolute abundance, and relative abundance of a multiplicity of species, genera, and other taxonomic units whose diversity, absolute abundance, and relative abundance are known to be associated with states of disease, disease risk, or health. The report (208) is then compared with a reference metric (210) established from studies of populations known to be in good health, or to be free of specific disorders known to be associated with a disrupted microbiome. The report (208) may also be compared with a reference metric (210) representative of populations known to have a specific disorder associated with a disrupted microbiome.

The comparison generates a specification (212) that identifies microorganisms found in elevated relative abundance and microorganisms found in depressed relative abundance. Optionally, the clinical sample is also subjected to laboratory determination of selected biomarkers (214) indicative of inflammation, organ dysfunction, and other indicators of disease or disease risk, and a biomarker report (216) is generated. The microbiome specification (212) and the biomarker report (216) are then transmitted (218) to a formulating facility (220).

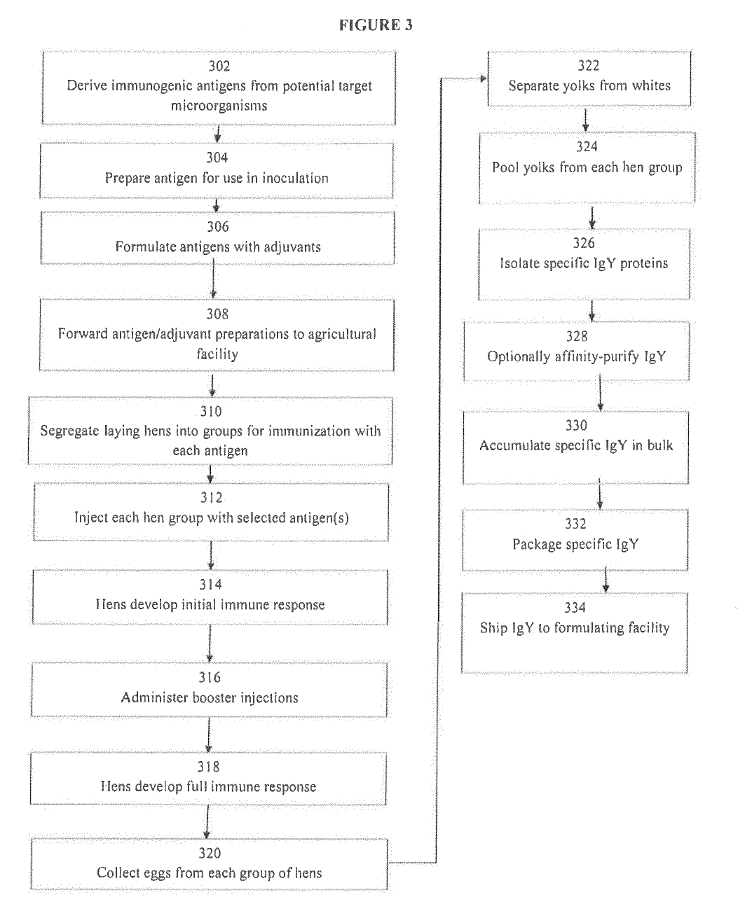

FIG. 3 illustrates a schematic of procedures for producing microorganism-specific IgY in bulk, pooling yolks and extracting specific IgY, affinity-purifying IgY as required, accumulating stocks of individual microorganism-specific IgY in gram, kilogram, or larger quantities, and shipping IgY to a formulating facility. It is understood that in practice the production, processing, storage, and final formulation of the IgY mixture may be done at a single facility, or at distributed facilities. As illustrated by FIG. 3, antigens (302) are derived from individual species of microorganisms known to contribute to disease or disease risk when present at elevated levels of relative abundance in a microbiome. Examples of such microorganisms are: a) in the Bacteroidetes phylum. Bacteroides vulgatus and Alistipes putredinis, b) in the Firmicutes phylum, Anaerotruncus coliformis, Butyrivibrio crossotus, Coprococcus eutactus, Faecalibacterium prausnitzii, Blautia coccoides, Ruminococcus torques, Veillonella atypica, and Enterococcus faecalis, c) in the Actinobacteria phylum, Bifidobacterium longum, Collinsella aerofaciens, d) in the Proteobacteriaceae phylum, Pseudomonas aeruginosa and Klebsiella pneumoniae. It is understood that additional target microorganisms in these and other phyla will be included as desired based upon their known associations with disease, disease risk, or health. It is further understood that functional microorganism-produced molecules may serve as antigens, producing IgY immunoglobulins targeting such molecules. Each antigen is prepared (304) for use in inoculating avian species, in this embodiment, laying hens, using techniques known to those skilled in the art. Each antigen is then mixed with appropriate adjuvant (immune-boosting agent) (306) known to those skilled in the art, and forwarded to an agricultural facility (308) dedicated to housing and care of groups of laying hens (310). At the agricultural facility, each group of laying hens is injected (312) with at least one antigen/adjuvant mixture, using techniques known to those skilled in the art. After an interval (314) that permits hens to develop an initial immune response (for example, 2 weeks), hens are inoculated with a booster injection (316) of antigen and adjuvant. After a second interval (for example, 7 additional weeks) (318), egg collection (320) begins. In a preferred embodiment, eggs collected from each group of hens are then processed to first separate yolks from whites (322); yolks from each group are then pooled (324), and yolks are then processed to isolate IgY protein from the yolks (326) using techniques known to those skilled in the art. In an optional step (328) IgY protein is affinity-purified to isolate concentrated IgY with specific affinity for the target microorganism(s) or functional microorganism-produced molecules. IgY protein (affinity-purified or not) is then accumulated (330) into gram, kilogram, or larger quantities, and packaged (332) for shipping (334) to the formulating facility (220) in FIG. 2.

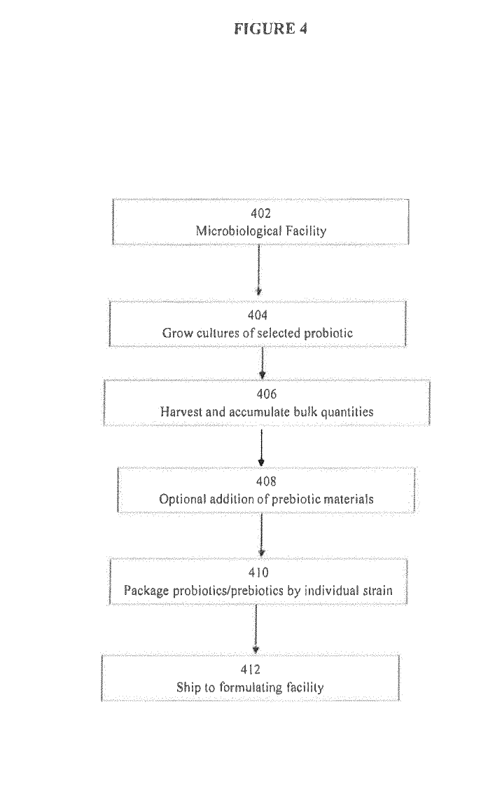

FIG. 4 illustrates a schematic of procedures, for raising bulk quantities of specific probiotic microorganisms at a microbiological facility, and then packaging them using a means that preserves their viability, for shipment to a formulating facility. As shown in FIG. 4, a microbiological facility (402) is selected, at which cultures of live probiotic microorganisms (404) known to contribute to disease or disease risk when present at depressed levels of relative abundance in a microbiome are grown, using techniques known to those skilled in the art. Using techniques known to those skilled in the art, bulk quantities (406) of individual species of such microorganisms are harvested and accumulated. Optionally, prebiotic mixtures (408) are added to individual bulk cultures of desired microorganisms to support their colonization and growth when administered to a host. Each individual culture of microorganisms is then packaged (410) in a fashion known to those skilled in the art for shipment (412) to the formulating facility (220) in FIG. 2. Examples of probiotic microorganisms and strains of said microorganisms known to be useful when administered as probiotics are: Bifidobacterium infantis 35624, Lactobacillus rhamnosus GG, and Lactobacillus plantarum 299v. Pre-determined mixtures or consortia of microorganisms may also be used, such as VSL #3, which contains cultures of Streptococcus salivarius species, Lactobacillus casei, plantarum, acidophilus, and delbrueckii subspecies bulgaricus, and Bifidobacteria longum, infantis, and breve. It is understood that additional probiotic microorganisms will be included as desired based upon their known associations with disease, disease risk, or health, and, furthermore, that techniques will be applied to preserve the viability of anaerobic organisms throughout the manufacturing and administration processes.

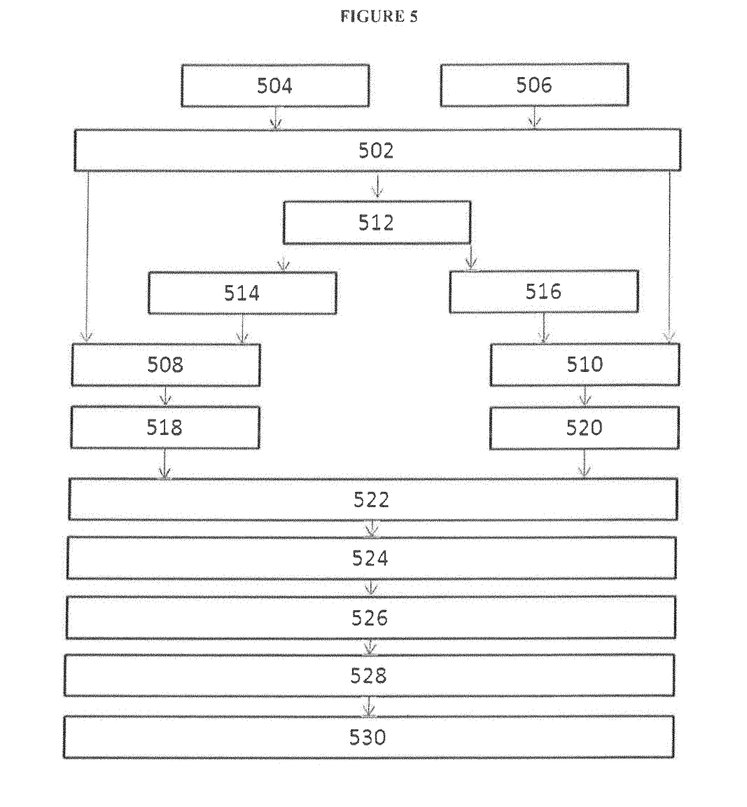

FIG. 5 illustrates the steps that will be taken at the formulating facility for preparation of each subject's or population's customized combination of microorganism-specific IgYs and live probiotic microorganisms. Both IgY and probiotic microorganisms may be prepared and packaged in forms that resist digestion or destruction in the upper gastrointestinal tract, to enhance their delivery to the colon, or in similar formulations, excipients, and so forth that enhance delivery to topical or mucosal sites. Both IgY and probiotic microorganisms may be formulated at different facilities, and shipped separately, should conditions require. It is understood that the processes described in FIG. 5, 504 (IgY) and 506 (probiotics) are separable, and that the final formulation for use in subjects may contain specific IgY(s), probiotic(s), or a combination of both components.

Actual calculation of the requisite amounts of IgY and probiotic microorganisms is amenable to computer algorithms or other automated means, and the calculation and instructions for formulation may occur at any physical location desired, so long as the final instructions are available at the formulation facility(ies).

As shown in FIG. 5, a formulating facility (502) receives bulk quantities of microorganism-specific IgY (504) from the agricultural facility (308) in FIG. 3, and bulk quantities of probiotic microorganisms (506) from the microbiological facility (402) in FIG. 4. The IgY and probiotic microorganisms are transferred to storage and dispensing areas (508) and (510), respectively. Upon receipt (512) of a subject's or a population's microbiome specification (220) from FIG. 2, a determination of the kinds and amounts of IgY required for subtractive therapy (514) and of the kinds and amounts of probiotic microorganisms required for additive therapy (516) is carried out by human or computer means, based upon the kinds and amounts of microorganisms found to be in higher or lower relative abundance in the specification (220). The results of these calculations are then transferred to the storage and dispensing areas (508) and (510), and, appropriate quantities of specific IgYs and probiotic strains (518) and (520) are prepared. Each preparation is then further processed (522), using techniques known to those skilled in the art, to be protected against upper gastrointestinal tract breakdown (e.g., liposomes, enteric coating, nanospheres, phytosomes, and others), or to be applicable to topical or mucosal sites. The mixtures are then packaged (524) and shipped to the subject (526), who ingests or applies the mixtures for a specified period of time to the site of the microbiome undergoing modulation. Clinical monitoring (528) is carried out to detect desired changes, and a repeat microbiome profile (530) is optionally obtained. The entire cycle may optionally be repeated until a desired clinical state is achieved, or until a desired microbiome profile is detected. Again, the processes of formulating IgY and pro/prebiotic components are severable, and may be applied independently or together.

### Characterization of a Microbiome:

In order to selectively modify a microbiome, it must first be characterized as to the microorganisms that comprise it, in a quantitative fashion that yields measurements of microorganism absolute and relative abundance, diversity, and the ratios of certain microorganism taxa to others.

Characterization of any microbiome is routinely done nowadays using metagenomic methods. Metagenomics refer to culture independent and sequencing-based studies of the collective set of genomes of mixed communities of microorganisms (metagenomes), permitting exploration of their compositional and functional characteristics. Currently, most metagenomic techniques rely upon molecular analysis of genetic material (ribosomal RNA, or rRNA) from the 16 S component of the 30 S small subunit of the prokaryotic ribosome (prokaryotes include all non-nucleated cells, including the bacterial microorganisms comprising a microbiome). 16 S rRNA contains, on the one hand, highly conserved primer binding sites, and also species-specific hypervariable regions. Detection and quantification of those species-specific regions is then used to produce a quantitative profile of microorganisms within a microbiome.

Molecular profiling of microbiomes via 16 S rRNA can be done by a variety of approaches, including terminal restriction fragment length polymorphism, polymerase chain reaction (PCR) temperature/denaturing gradient gel electrophoresis, and fluorescent in situ hybridization. DNA microarrays and next-generation sequencing (NGS) technologies may now be applied to study relevant aspects of microorganism ecology, including total diversity and a range of biochemical functions. NGS approaches, including pyrosequencing and other platforms such as Solexa (Illumina, Inc.) and SOLiD (ABI, Inc.) provide rapid and highly parallel sequencing of many DNA fragments from complex samples; other platforms have also recently been adopted in this field and are known to those skilled in the art. The increasing availability and falling costs of techniques not limited to ribosomal RNA/DNA analyses has made it possible to assess the full genome of most microorganisms (whole genome sequencing, shotgun sequencing), which also results in the ability to identify individual species and even strains present in a microbiome. Essentially any technique known in the art capable of providing qualitative and quantitative information about the microbial population making up a given microbiome will be appropriate to provide the information needed in this step.

Because there are thousands of species in most microbiomes, it has become commonplace to measure and report only a selected number of species for research and clinical use. A variety of genera, strains, species, and operational taxonomic units (OTUs) has been identified that contribute heavily to both the variability of a given microbiome, and to its relative contribution to states of health or increased disease risk.

Microbiome profiles can now be obtained from a variety of commercial diagnostic laboratories that use techniques similar to those described above. Such profiles generally consist of a report detailing the absolute and relative abundance of a set of microorganism targets selected from those groups with known associations with health, disease, and disease risk. Such reports may also include a measure of the diversity of the microbiome, because in general microbiomes with greater diversity of microorganisms are associated with better health and greater resistance to disease. Another measure associated with certain diseases or disease risk is the ratio of the abundance of specific taxa, the best-known of which is the ratio of abundance of microorganisms in the Firmicutes phylum to that of microorganisms in the Bacteroidetes phylum, sometimes known as the “F/B” ratio.

With increasing availability of whole genome sequencing and the like techniques, microbiome profiles now provide information about microorganisms in a microbiome beyond that provided by targeted techniques such as 16 S sequencing, expanding the utility of the effort to characterize microbiome compositions in great detail. Such techniques can identify individual species and even strains having close statistical associations with states of disease or disease risk. This capability increases the need in the field for research and therapeutic interventions capable of acting at a similarly-detailed level of specificity, that is, at the level of species or strains.

Studies of large numbers of individuals and body sites have demonstrated that certain patterns of microbiome disruptions are strongly associated with certain states of disease, disease risk, or health. Characteristics of the microbiome associated with such risks include (but are not limited to) alterations in the relative abundance (RA) of individual species, genera, families, phyla, or other OTUs, reductions in the overall diversity of the microorganisms represented, and the Firmicutes/Bacteroidetes ratio. An elevated F/B ratio has been identified in a number of states of disease or disease risk, including but not limited to IBS and cardiometabolic disease.

### Production of IgY for Subtractive Therapy:

This invention discloses the use of specially-prepared mixtures containing at least one IgY specific of at least one taxonomic level of microorganism found to be in increased relative abundance in a microbiome, with the aim of reducing the relative abundance of said at least one microorganism in a microbiome, or containing at least one IgY specific of at least one functional microorganism-produced molecule with the aim of reducing or eliminating said at least one functional microorganism-produced molecule.

The production of IgY from domestic chicken eggs is known to those skilled in the art. In its essence, it involves selection of a healthy laying hen, and selectively injecting that hen, subcutaneously or intramuscularly, with an antigen of interest. Over a several-week period, the hen's immune system responds to the antigen by producing large amounts of IgY, which become concentrated in her eggs. A booster injection is commonly given between weeks 2 and 4 to enhance immunoglobulin production, and full immunoglobulin production is reached at around week 10-12. A laying hen produces on the order of 350 eggs/year, each egg containing 100-200 mg IgY. Production rates vary by breed or genetic lines, as well as on a septadian biorhythm. Eggs are collected, yolks are separated from whites, and pooled from multiple hens having received the same antigen injection, and then, as is known by those skilled in the art, undergo a small number of physical and chemical steps for purification and extraction of the IgY proteins. Because yolks of immunized hens also contain naturally-occurring IgY, the extracted IgY proteins may be further affinity-purified, to selectively harvest only the specific IgY desired for therapeutic use. Alternatively, the IgY proteins may be administered without affinity-purification, so long as sufficient amounts of the active IgY are found in the product to achieve the goals of subtractive therapy.

Specific details, including preparation and optimization of antigen doses, choice of adjuvant (immune-boosting material), immunization interval, and techniques for isolating and purifying IgY, are known by those skilled in the art and are available in the scientific literature.

In one embodiment of the present invention, IgY will be generated against a group of microorganisms known to be associated with impairments of health, or increased disease risk, when found in relatively high abundance in a microbiome. Known as “subtractive therapy,” this approach has been realized only partially, with the use of antibiotics to which only certain large classes of microorganisms are sensitive. Antibiotics carry with them a host of burdens, including the fact that they are never specific to an individual species of microorganism, they can only be effective against bacteria, not viruses, yeasts, fungi, and other non-bacterial microorganisms, they cannot readily be titrated to effect (so that they either eradicate or spare populations, rather than modulating them), and they rapidly select for antibiotic-resistance not only in target microorganisms, but also in others not necessarily associated with an individual impaired microbiome. Indeed, a risk of antibiotic approaches to subtractive therapy is the destruction of microorganisms whose presence contributes to a health-associated microbiome. Furthermore, antibiotic use is itself a frequent cause of a disrupted microbiome, or dysbiosis.

Once a sufficient quantity of IgY has been prepared against microorganisms known to be associated with disease or disease risk when found in high relative abundance, selected kinds and quantities of IgY will then be prepared for oral administration to a subject whose microbiome has been characterized, or to a group of subjects having in common a disease or a disease risk, and for whom a representative microbiome profile is established. The administered IgY has been shown to survive passage through the upper gastrointestinal tract in quantities sufficient to bind to microorganisms in the colon. Should greater quantities of IgY be required to achieve the desired subtractive effect, techniques for preventing destruction of ingested proteins such as IgY (e.g., liposomes, nanospheres, and others), which are known to those skilled in the art, may be used in formulation to protect the active IgY.

### Specific Embodiments of the Invention:

Many embodiments of the present invention are possible.

In one embodiment, the process includes application of avian egg yolk-derived immunoglobulins (IgY) as selective, specific, modifiable, and reversible agents to reduce individual microorganism species' abundance in the microbiome, or to reduce the amounts of functional microorganism-produced molecules present at a microbiome site.

In this embodiment, a microbiome profile is generated as described above, using as its source a biological sample taken from a body site in which a relationship between a microbiome profile and a disease or disease risk is known. Metagenomic data on the microbiome profile are used to determine which species, genera, or other OTUs are found in excessively high relative abundance, or contribute to an elevated F/B (or other relevant) ratio, when compared with a reference value. It is important to note that existing technology supports not only qualitative determination of microorganisms in a given microbiome, but also quantitative determination of each microorganism's absolute abundance, in units familiar to microbiologists, e.g., in the equivalent of colony-forming-units (CFUs).

With metagenomic data identifying microorganisms present in high relative abundance, or that are known to produce functional microorganism-produced molecules in hand, IgY is prepared as described above, in such a fashion as to produce a therapeutic amount of at least one IgY specific of at least one microorganism found to be in elevated relative abundance in a microbiome, or at least one IgY specific of at least one functional microorganism-produced molecule.

The therapeutically effective amount of at least one IgY is administered to at least one subject, or at least one member of at least one group of subjects having in common a disease or disease risk, thereby reducing the relative abundance of at least one microorganism or at least one functional microorganism-produced molecule, in the microbiome of the at least one subject. IgY prepared for use in this invention will be targeted against at least one strain, taxon or operational taxonomic unit known to be associated with disease or disease risk when present at high relative abundance in the microbiome of the at least one subject.

The microbiome profile used to specify the production of the at least one IgY is obtained by metagenomic analysis of at least one microbiome sample obtained from at least one body site selected from the group consisting of intestinal, gastric, oral, oropharyngeal, otic, nasal, nasopharyngeal, skin, axillary, vaginal, and conjunctival microbiomes.

The at least one microorganism targeted by the administered therapeutically effective amount of IgY is at least one non-pathogen selected from the group consisting of bacteria, fungi, yeasts, viruses, prions, parasites, and other life forms comprising a microbiome of the subject. It is equally possible to target the therapeutically effective amount of IgY against at least one pathogen selected from the group consisting of bacteria, fungi, yeasts, viruses, prions, parasites, and other life forms with pathogenic potential in the subject.

It is anticipated that IgY subtractive therapy will be targeted against at least one microorganism that is associated with a specific disease or disease risk when present at a relative abundance higher than a reference value.

It is further anticipated that the relative abundance of the at least one targeted microorganism will be reduced as a result of IgY administration.

In some embodiments, the relative abundance of the at least one targeted microorganism is reduced.

As noted above, the IgY administered to the subject or group of subjects may contain at least one IgY targeting at least one functional microorganism-produced molecule known to be associated with disease or disease risk. Examples of such pathogenic microorganism-produced molecules include, but are not limited to, lipopolysaccharides (LPS), flagellins, adhesion factors, endotoxins, exotoxins, verotoxins, virulence factors, bacteriocins, or peripheral membrane proteins. Such IgY may be administered together with, or separately from, the IgY mixture targeting microorganisms.

It is anticipated that the at least one IgY will be administered to the at least one subject, or the at least one member of a defined population having in common a disease or a disease risk, at least once per day and for at least 2 days, to permit delivery of a steady amount of IgY to the targeted microbiome site. In a one embodiment, the target microbiome is that of the gastrointestinal (GI) tract, and in that embodiment the route of administration of the IgY is oral. In other embodiments, however, the target microbiome is one of those iterated above, and the route of administration of the IgY is selected from the group consisting of oral, enteric, intraoral, intraduodenal, intragastric, intravaginal, intra-otic, and topical.

Following administration of the first dose of the therapeutically effective mixture of IgY to a subject, the subject may be monitored for a therapeutic effect. Such an effect may be represented by a change in manifestations of disease or disease risk (for non-limiting example, an improvement in symptoms), by a change in the presence of a biomarker relevant to a disease or disease risk (for non-limiting example, a reduction in fecal calprotectin, an indicator of intestinal inflammation), by a change in microbiome diversity or relative abundance of at least one microorganism, or by a change in the presence or amount of at least one functional microorganism-produced molecule.

As a result of such monitoring, which may comprise repeated measurements of the microbiome profile, symptom severity scores, or biomarkers associated with disease or disease risk, treatment with the therapeutically effective amount of IgY may be modified (for non-limiting example, by increasing the amount of IgY targeting a particular microorganism whose relative abundance remains elevated), or terminated.

In another embodiment, a mixture of IgYs specific of at least one microorganism, or of at least one pathogenic microorganism-produced molecule, is formulated in response to a microbiome profile of an individual subject, or one representative of a defined population of subjects having in common a disease or a disease risk, that identifies at least one microorganism to be present at a relative abundance higher than a reference value. In some embodiments, a mixture of IgYs specific of at least one microorganism, or of at least one functional microorganism-produced molecule, is formulated in response to a microbiome profile of an individual subject, or one representative of a defined population of subjects having in common a disease or a disease risk, that identifies at least one microorganism in higher relative abundance when compared to a normal reference.

Similarly, the IgY mixture may contain IgY specific of at least one functional microorganism-produced molecule. In this embodiment, the mixture of IgYs is customized (to the individual or representative group microbiome profile) to target and reduce the abundance of the at least one targeted microorganism, or the presence of the at least one functional microorganism-produced molecule.

In this embodiment, determination that at least one microorganism is present in a microbiome is elevated is performed by comparison of the subject's microbiome profile, or the group's representative microbiome profile, with a reference value.

In this case, microorganisms found to be present in relative abundance higher than a reference value will be targeted by the production of microorganism-specific IgY.

Alternatively, or additionally, the subject's or the group's microbiome profile may be compared with a microbiome profile representative of a population of subjects having in common a disease or a disease risk. In this case, IgY is prepared against microorganisms found to be present in relative abundance similar to, or elevated, when compared with the known diseased or at-risk-for-disease population.

These comparisons can be made by at least one of a number of computational and statistical methods known to those skilled in the art. Non-limiting examples of such methods include calculation of difference scores, application of principle component analysis, and multiple regression analyses.

In one embodiment, IgY may also be prepared targeting at least one functional microorganism-produced molecule known to be associated with a specific disease or disease risk. Similarly, IgY may be prepared targeting at least one functional microorganism-produced molecule known to be associated with at least one microorganism found to be present in increased relative abundance on the microbiome profile of at least one subject, or in the representative microbiome profile of a group of subjects having in common a disease or a disease risk.

In one embodiment, the customized, therapeutically effective mixture of IgYs is formulated from a stock of individual IgYs, each specific of one microorganism or one functional microorganism-produced molecule.

In another embodiment, instead of formulation of the IgY mixture from separate stores of individual IgYs specific of individual microorganisms, the mixture is formulated in vivo by immunization of a group of hens against more than one microorganism in a microbiome, or by immunizing hens against more than one functional microorganism-produced molecule. In this embodiment, all eggs from the similarly-immunized group of hens contains the same customized mixture of IgYs. This method may be particularly expedient for production of fixed or standardized mixtures of IgYs aimed at members of populations having in common a disease or a disease risk.

The customized mixture of IgYs produced in any one of these fashions is formulated in a way calculated to reduce the relative abundance of the targeted at least one microorganism towards its relative abundance on a reference value.

In some embodiments, the customized mixture of IgYs is formulated in a way calculated to reduce the relative abundance of the targeted at least one microorganism.

In one embodiment of this invention, the customized mixture of IgYs is packaged in a pharmaceutically acceptable protective agent capable of preventing degradation of the IgY proteins in the stomach or small intestine.

In one embodiment of the invention, the at least one subject is a human.

In another embodiment, the at least one subject is an animal selected from the group consisting of: a primate, a rodent, a feline, a canine, a poultry, an avian, a Bovid, a Suid, an Ovid, or another domesticated animal.

In various embodiments of this invention, the at least one subject may be a member of a defined population having in common a disease or risk for a disease. In such embodiments, the microbiome profile used to specify the customized therapeutic mixture of IgYs is derived from representative samples of the microbiomes of such a defined population existing in published literature or individual databases.

In one embodiment of the invention the therapeutically effective amount of IgY is administered to at least one subject who has, or is at risk for having, at least one disease selected from the group consisting of irritable bowel syndrome, inflammatory bowel disease, other functional bowel disorders, celiac disease, non-celiac gluten sensitivity, small intestinal bacterial overgrowth, migraine, cardiovascular disease, type 1 or type 2 diabetes, obesity, the metabolic syndrome, osteoporosis, autoimmune disease, osteoporosis, behavioral disorder, malignancy, and psychiatric disorder.

In certain embodiments, it is advantageous not only to reduce the relative abundance of specific microorganisms using IgY subtractive therapy, but also to increase the relative abundance of other specific microorganisms, in an effort to restore a microbiome to a state more closely associated with health. In such embodiments, according to this invention, a microbiome profile is used to identify which microorganisms are present in a state of low relative abundance, and to use that microbiome profile to guide the formulation of a mixture containing at least one living probiotic species or strain of those microorganisms in order to produce an increase in their relative abundance in the microbiome. It is understood that this approach may be applied to an individual subject, based on that subject's own microbiome profile, or to a population of subjects having in common a disease or a disease risk, based on a representative microbiome profile of a similar group of subjects, for example, existing in published literature.

In these embodiments, the probiotic microorganism is comprised of at least one microorganism found in the microbiome of an individual subject or in a population of subjects having in common a disease or a disease risk. Additionally, because certain strains of probiotic microorganisms not normally found in a microbiome are known to produce desirable increases in the relative abundance of desirable microorganisms in a microbiome when administered, the probiotic mixture may be comprised, entirely or in part of at least one such probiotic microorganism.

It is understood that this invention may be applied to any body site in which a microbiome associated with health at that site, or elsewhere in the body, occurs naturally. Such microbiomes may include any microbiome site selected from the group consisting of intestinal, gastric, oral, oropharyngeal, otic, nasal, nasopharyngeal, skin, axillary, vaginal, and conjunctival microbiomes.

The object of these embodiments of the invention is to favorably raise the relative abundance of specific microorganisms in a microbiome towards a reference value.

It is estimated that application of the invention will result in an increase of those specific microorganisms. It is understood that, when the probiotic is comprised of special species or strains not normally present in the microbiome of the subject or group of subjects, its administration will result in an increase of desirable microorganism in the microbiome by about the same range. Ongoing experiments will be conducted to determine the optimum doses of probiotics required to produce increases in relative abundance in this range.

Regarding the nature of the administered probiotic strains or species of microorganism, it may be advantageous to use a probiotic species that is itself present at low relative abundance in a microbiome. It may also be advantageous to administer a probiotic microorganism species or strain that is not itself a normal member of a microbiome, but which has been shown, on administration to a microbiome, to result in increased relative abundance of microorganisms found in the microbiome at reduced relative abundance in a fashion known to be associated with disease or disease risk.

As is understood by those skilled in the art, the probiotic species administered in this invention are produced by specific culture methods favoring the growth of desired probiotic microorganisms. These microorganisms are administered in a therapeutically effective amount calculated to raise the relative abundance of strains or taxa of microorganisms known to be associated with disease or disease risk when present at low relative abundance in a microbiome. In some embodiments, these microorganisms are not themselves present in a microbiome, but are known to increase the relative abundance of such microorganisms that are associated with disease or disease risk when present at a reduced relative abundance in a microbiome.

It is anticipated that the probiotic mixture will be administered at least once daily to a subject or group of subjects, for a period of at least two days, to ensure adequate increases in the desired microbiome microorganisms. In most embodiments of the invention, the probiotic mixture is administered by a route selected from the group consisting of oral, enteric, intraoral, intraduodenal, intragastric, intravaginal, otic, and topical.

It is advantageous to monitor a subject or members of a population of subjects having in common a disease or a disease risk, to detect changes in biological characteristics that may arise from administration of the probiotic mixture. Such changes include, but are not limited to, alterations in manifestations of a disease or disease risk, changes in biomarkers relevant to a disease or a disease risk, and microbiome characteristics including, but not limited to, changes in diversity, relative abundance, or absolute abundance of microorganisms of interest, in comparison to pre-treatment values. It will be advantageous to use the results of such monitoring to make necessary changes in the duration of use, or in the dosing, of the probiotic formulation. In some embodiments, changes may include termination of treatment with the probiotic formulation.

In one embodiment of this invention, a therapeutically effective mixture of probiotics specific of at least one microorganism in a microbiome is prepared, guided by a microbiome profile obtained from a body site of a subject or a group of subjects having in common a disease or a disease risk. With such metagenomic data in hand, such a mixture can be readily formulated by including in it a therapeutically effective amount of specific microorganisms found to be present at low relative abundance in the microbiome profile. It may also be advantageous to use as the probiotic at least one microorganism species or strain that is not naturally found in a microbiome, but is known to produce an increase in the relative abundance of microorganisms found to be present at low relative abundance in a microbiome, such microorganisms being known to be associated with disease or disease risk when present at low relative abundance in a microbiome.

In one embodiment, the microbiome profile of an individual subject, or a microbiome profile representative of a group of subjects having in common a disease or a disease risk, reveals that at least one microorganism in the profile is present in decreased relative abundance in comparison with a normal reference microbiome representative of subjects in good health. Alternatively, the microbiome profile of the subject or that representative of a group of subjects is compared with a microbiome profile representative of a group of subjects with a known microbiome-associated disease or disease risk, such as but not limited to IBS. In this case, the comparison may reveal at least one microorganism to be present at similar or reduced abundance to that seen in the microbiome profile representative of those with the disease or disease risk. In both cases, the therapeutically effective mixture of probiotic microorganisms is prepared in a fashion intended to increase the relative abundance of the microorganisms of interest when applied to the site of the microbiome.

In some embodiments, the customized probiotic mixture is prepared from existing stocks of living probiotic microorganisms, in order to permit either personal customization or customization suitable for raising the relative abundance of specific microorganisms in members of a group of subjects having in common a disease or a disease risk. Such a preparation method also enables modification of any one mixture of probiotics to meet the changing needs of a subject or group of subjects.

Formulation of the customized mixture of probiotic microorganisms will be done using doses of individual probiotic microorganisms calculated to increase the relative abundance of each targeted microorganism from about 1% to about 100% of the total abundance of the microorganism on the original microbiome profile. In a preferred embodiment, the doses of individual probiotic microorganisms are selected with the aim of increasing relative abundance of targeted microorganisms by a range of about 10% to about 40% of the abundance of the microorganism of interest in the original microbiome profile. Experiments will be conducted to derive estimates of probiotic doses required to achieve increases in relative abundance in these ranges.

In other embodiments, the probiotic microorganism mixture is packaged with a pharmacologically acceptable protective agent to permit delivery of the mixture to the lower gastrointestinal tract.

It is understood that the invention as disclosed herein may be used with individual human subjects, or with human subjects who are members of a group having in common a disease or a disease risk, and that the subject or group of subjects may also be animals selected from the group consisting of: a primate, a rodent, a feline, a canine, a poultry, an avian and other domesticated animals.

It is also understood that the microbiome profile used to specify the formulation of the probiotic mixture may be one representative of a defined population having in common a disease or a disease risk.

In many embodiments of this invention, the relative abundance of the microorganisms of interest is determined to be reduced when compared with a microbiome profile representative of a healthy population. In addition, or alternatively, the microorganisms of interest may be found to be present in a microbiome of a subject, or the microbiome profile representative of a group of subjects, at a relative abundance similar to or reduced when compared with a microbiome profile representative of a group of subjects with a known disease or disease risk.

This invention is understood to be applicable to at least on subject, or any individual member of a group of subjects, having or being at risk for having at least one disease selected from the group consisting of irritable bowel syndrome, small intestinal bacterial overgrowth, inflammatory bowel disease, other functional bowel disorders, celiac disease, non-celiac gluten sensitivity, migraine, cardiovascular disease, type 1 or type 2 diabetes, obesity, the metabolic syndrome, osteoporosis, autoimmune disease, osteoporosis, behavioral disorder, malignancy, and psychiatric disorder.

In one embodiment, the invention involves a process by which a microbiome profile report of an individual or one representative of a group of individuals having in common a disease or a disease risk is used to inform production of mixtures of IgY immunoglobulins targeted against microorganisms found in high relative abundance compared with that found on microbiome profiles of healthy populations. Alternatively, the reference microbiome profile is one representative of a group of subjects having in common a disease or disease risk, in which the relative abundance of specific microorganisms is known to be reliably elevated. The resulting IgY immunoglobulin mixture is administered to the end-user, an individual whose microbiome is in need of modification. The end-user consumes or applies the IgY mixture for a pre-determined period ranging from 2 days to longer periods. A repeat microbiome profile may be obtained during or after the treatment period, to monitor clinical progress and alterations in the microbiome, and this information may be used to modify or cease IgY therapy.

In another embodiment, the IgY may be produced against non-bacterial microorganisms (e.g., viruses, fungi, yeasts, prions), or against naturally-occurring functional molecules (e.g., wheat gliadin), or functional microorganism-produced molecules, instead of or in addition to the bacterial microbiome components.

In yet another embodiment, the IgY formulation may be prepared for topical subtractive therapy by administration (e.g., to skin sites), local irrigation (e.g., to oral, nasal or vaginal mucosa), again with the specific IgY raised against microorganisms found in excessive abundance for a given microbiome site.

In still another embodiment, any of the preceding processes may be augmented by additive therapy, in which live biotherapeutic cultures or consortia of microorganisms are provided in addition to the IgY subtractive products. Such probiotic microorganisms would, again, be titrated in amounts determined by future experimentation to achieve the optimal abundance of each microorganism in the microbiome. Such an embodiment would include both customized formulations of IgY and live biotherapeutics created on the basis of personal microbiome profiles, and those created on the basis of microbiome profiles typical of populations of subjects having in common a disease or a disease risk.

In still another embodiment, prebiotic materials may also be added to the formulation, in order to selectively nourish desired microorganisms and/or foster production of natural substances (e.g., bacteriocins) by such microorganisms that work to suppress populations of less desirable microorganisms.

In a different embodiment, preparations of at least one target-specific IgY may be used in research on the relationships between microbiome composition and manifestations of disease, disease risk, or health. Such preparations can be administered to living experimental animals by customized addition to feed (e.g., pellets), or in a vehicle such as phosphate-buffered saline (PBS) for gavage. Similarly, such preparations may be added to experimental model microbiomes. In all such cases, researchers would monitor the microbiome of interest for changes in abundance of the targeted microorganisms or functional microorganism-produced molecules, and correlate those changes with alterations in phenotype, disease manifestations, biomarkers of disease or disease risk, biometric measures, and other metrics used to assess health, disease, or disease risk.

In another embodiment, target-specific IgY is used in an antigen-detection system (ELISA or similar) for identifying the abundance of selected microorganisms in a microbiome specimen. Such a system is advantageous because it delivers rapid results and will not require extraction of DNA or RNA as is done in molecular detection systems. For non-limiting example, a test strip can be constructed in which IgY targeting the nine microorganisms identified as being over-abundant in irritable bowel syndrome (IBS) are used. Clinicians or end-users could then determine the abundance of said microorganisms in an individual specimen at baseline, and could follow the abundance of said microorganisms during a course of treatment aimed at altering abundance of microorganisms.

### Advantages of the Invention Include:

Use of IgY for subtractive therapy (reducing relative abundance of specific microorganisms in microbiome) is microorganism-specific, allowing only selected species or other taxonomic units to be reduced in quantity. This is an advantage over all other known subtractive therapies, which can only subtract or delete larger, and non-taxonomically-defined, classes of microorganisms in a broad fashion. For example, antibiotics are not species-specific, and can only be targeted at larger classes of microorganisms in an aim to reduce the abundance of the entire Firmicutes phylum, though the high relative abundance of Firmicutes is rarely caused by elevation of all members of that phylum in a given microbiome. Rather, such an elevation is typically produced by high relative abundance of specific microorganisms within the phylum Firmicutes. IgY molecules are capable of targeting individual offending species, while leaving intact other members of the Firmicutes phylum that are not directly contributory to its elevated relative abundance.

Subtractive therapy with IgY will also allow calibrated, or titrated removal of microorganisms, recalling that none of the microorganisms that contribute to imbalance in the microbiome are explicitly pathogens, so that complete removal of such microorganisms is distinctly undesirable. Other subtractive therapies are “all-or-nothing” in their targeting.

A related advantage is that continuous or intermittent re-dosing of IgY subtractive therapy can be changed to adapt to changes in a microbiome. Thus, over-shooting or undershooting targets' desired relative abundance is readily corrected, and, as an individual's biology, environment, diet, and other factors change, the IgY therapy can be modified accordingly, so that a constant balancing force is applied to the microbiome.

While antibiotic resistance is a known and widespread threat to public health as well as health of the individual, it is not expected, and has to date not been shown, that use of IgY against bacterial targets produces resistance, and, in fact, the mechanisms by which IgY operate to destroy or remove selected species depend only on evolutionarily-determined surface markers, not on any adaptable physiology of the target microorganism. Moreover, should bacteria in fact be naturally selected to reduce or eliminate display of target molecular markers in response to persistent IgY therapy (at present an entirely theoretical risk), the nature of IgY production makes it possible to choose from any other identifying molecular feature of the microorganism, thereby renewing the ability to identify and subtract it from the population.

IgY subtractive therapy is advantageous over fecal microbial transplants as well, again because of its specificity of targeting and the ability to continuously modify the treatment. FMT may also carry substantial risks of infection of the recipient, a risk not associated with IgY

No genetic modification of any living microorganism is required for IgY production or administration in microbiome modification, unlike recently-disclosed approaches that rely on engineered microbes to control, amplify, or subtract from the healthy microbiome. Such engineered approaches carry with them the real threat of environmental release of microorganisms with synthetic properties and unknown consequences.

IgY has now been developed to target not only microorganisms, but specific functional molecules. Unlike other subtractive therapies to date, this may allow IgY to be used against bacterial toxins, lipopolysaccharides, and other microbe-generated molecules that cause damage to host tissues, or facilitate interactions with other microorganisms that are deleterious to the host.

Production of IgY is relatively simple and non-invasive, compared to current methods of producing immunoglobulins for human experimental or therapeutic purposes. IgY production may require as few as two injections per hen. Hens may be housed humanely under cage-free conditions, and would be expected to have lifespans comparable with those of other domestic fowl.

IgY can be produced in polyvalent mixtures from a single hen, to boost productivity. In this fashion, a hen may be immunized to an arbitrarily large number of antigens, resulting in eggs enriched in not one, but several or many IgY molecules.

IgY production is relatively inexpensive, compared to most other technology endeavors; it involves essentially standard animal husbandry, including immunization of individual hens. Egg collection, separation, and processing can also be done using existing food-service technologies.

Furthermore, the rapid pace with which new IgY molecules can be produced is expected to improve efficiency and shorten discovery periods for therapeutic mixtures. Should an initial formulation prove less effective than hoped, it can be rapidly re-formulated with new IgY targeting different epitopes, microorganisms, or microorganism-produced molecules, and then rapidly subjected to renewed testing.

Subtractive therapy of any kind, including IgY, is advantageous over existing means of manipulating the microbiome, which to date are limited largely to dietary changes, probiotics, and prebiotics, none of which appear, alone, to produce lasting changes in the microbiome. The addition of IgY subtractive therapy to additive therapy such as pre- and probiotic therapies may result in improved persistence of the added microorganisms, and may contribute favorably to increased microbiome diversity, a desirable change.

Performing all of the steps outlined in FIG. 1 and described in the body of the disclosure is advantageous because it will contribute to efficiency and reduce error rates.

When applied for research purposes, use of subtractive approaches using IgY targeting individual species, strains, or other taxonomic units in an experimental animal's microbiome, or in a laboratory model microbiome, offers the advantage of specific intervention at desired taxonomic levels, followed by observation and measurement of resulting changes in microbiome composition and function. Intervention at such specific levels is not currently possible, limiting the amount of information that can be derived from microbiome composition-function studies.

### Unique Elements of the Invention Include:

Application of IgY technology to regulation of the microbiome (with the advantages enumerated above).
Provision of a means of “fine tuning” the microbiome by titrating or calibrating doses to observed effects.
Low cost.
Rapid response times to observed microbiome imbalances.
Rapid revision of test formulations to achieve optimal results prior to marketing or human testing.
A personalized approach that matches each individual's microbiome profile, across dimensions of time and space, allowing continuous revision and modification of individual therapy.
An approach that is not theoretically limited to specific classes of microorganisms (e.g., non-beta-lactamase producers, as in certain antibiotics), but rather can be applied to all known existing microorganisms as well as those discovered in the future.
An approach that is not limited to prevention or treatment of any one disease or disease process (of patents and applications by Turnbaugh, Kaplan, and Blaser, which are restricted to microbiome modification for prevention or treatment of obesity and obesity-related disorders).
An experimental tool (target-specific IgY) that permits selective removal or reduction in abundance of targeted strains, species, or other taxonomic units as part of studies of microbiome composition-function studies.

### Kits:

The invention includes a kit comprising at least a compound of the invention, an applicator, and an instructional material for use thereof. The instructional material included in the kit comprises instructions for preventing or treating a disease associated with characteristics of a microbiome in a subject. The instructional material recites the amount of, and frequency with which, the at least one compound of the invention should be administered to the subject. In other embodiments, the kit further comprises at least one additional antitumor agent.

### Administration/Dosage/Formulations:

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The therapeutically effective amount or dose of a compound of the present invention depends on the age, sex and weight of the patient, the current medical condition of the patient and the progression of a disease or disorder contemplated in the invention.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

Suitable doses of a compound of the present invention may vary across a wide range of values, depending on the degree of decrease or increase in the relative abundance of targeted microorganisms desired. The dose may be administered in a single dosage or in multiple dosages, for example from 1 to 4 or more times per day. When multiple dosages are used, the amount of each dosage may be the same or different. For example, a dose of 1 g per day may be administered as two 0.5 g doses, with about a 12-hour interval between doses.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In one embodiment, patients require intermittent treatment on a long-term basis upon any recurrence of symptoms.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is preferable to include isotonic agents, for example, sugars, sodium chloride, or polyalcohols such as mannitol and sorbitol, in the composition.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for any suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., analgesic agents.

Suitable compositions and dosage forms include, for example, dispersions, suspensions, solutions, syrups, granules, beads, powders, pellets, liquid sprays for nasal or oral administration, dry powder or aerosolized formulations for inhalation, and the like. It should be understood that the formulations and compositions that would be useful in the present invention are not limited to the particular formulations and compositions that are described herein.

### Oral Administration:

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds of the invention, and a further layer providing for the immediate release of a medication for treatment of a disease or disorder. Using a wax/pH-sensitive polymer mix, a gastric insoluble composition may be obtained in which the active ingredient is entrapped, ensuring its delayed release.

### Parenteral Administration:

For parenteral administration, the compounds may be formulated for injection or infusion, for example, intravenous, intramuscular or subcutaneous injection or infusion, or for administration in a bolus dose and/or continuous infusion. Solutions, suspensions or emulsions in an oily or aqueous vehicle, optionally containing other formulatory agents such as suspending, stabilizing and/or dispersing agents may be used.

### Additional Administration Forms:

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 2003/0147952, 2003/0104062, 2003/0104053, 2003/0044466, 2003/0039688, and 2002/0051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 min up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 min, about 20 min, or about 10 min and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 min, about 20 min, or about 10 min, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

### Producing Type-Specific IgY Against Selected Microorganisms and Determining Target-Specificity and Growth Inhibition

Materials and Methods: Pure live cultures of representative species of microorganisms known to be found in elevated relative abundance in two human disease states were obtained from commercial sources. In this example, the disease states are irritable bowel syndrome (IBS), and diet-induced obesity (DIO).

Published studies of IBS were first reviewed in search of examples of microorganism species, genera, families, or higher taxonomic units found in at least two studies to be associated with at least one of the disorders of interest when identified in significantly greater abundance in diseased subjects compared with those free of the disease or disease risk. When individual species were identified in such studies, those species were included; when only genera, families, or higher taxonomic units were reported, type species or commonly-available species were selected.

In the case of DIO, whole-genome shotgun sequencing was performed on pooled fecal pellets from 3 lean and 3 DIO C57/B6 mice to obtain comprehensive measurements of all identifiable microorganisms in each microbiome. Analysis was conducted to identify species in substantially greater abundance in the DIO compared with the lean group of mice. These organisms were then determined to be targets for reduction using selectively targeted IgY.

In this example, for IBS, the following nine microorganism species, representing taxa known to be associated with IBS when present in elevated abundance in a human fecal microbiome, were selected based on published studies: Alistipes putredinis (Weinberg et al.) Rautio et al. (ATCC® 29800™), Bacteroides ovatus Eggerth and Gagnon (ATCC® 8483™), Blautia [Clostridium] coccoides (ATCC® 29236™), E. coli Castellani and Chalmers (ATCC® 12435™), Pseudomonas aeruginosa (Schroeter) Migula (ATCC® 47085™), Ruminococcus torques Holdeman and Moore (ATCC® 27756™), Veillonella atypica (Rogosa) Mays et al. (ATCC® 27215™), Klebsiella pneumoniae (Schroeter) Trevisan (ATCC® 13883™), Enterococcus faecalis Schleifer and Kilpper-Balz (ATCC® 19433™).

In this example, for DIO, the following six microorganism species, representing taxa known to be associated with cardiometabolic disease when present in elevated abundance in a human fecal microbiome, were selected based the whole genome shotgun sequencing process: Ruminococcacae bacterium D16 strain KNHs216 (ATCC® TSD-27, Lachnospiraceae bacterium 28-4 strain KNHs209 (ATCC® TSD-26), Clostridium difficile CD1655 (ATCC® BA2826), Bacteroides thetalotaomicron UN3373 (ATCC® 29741). Bacteroides ovatus Eggerth and Gagnon (ATCC® 8483™), and Lactococcus lactis subspecies cremoris (ATCC® 19257).

The total number of microorganisms identified for use in this experiment is fourteen, comprised of nine in the IBS group and six in the DIO group, with one microorganism, Bacteroides ovatus, common to both groups.

For this experiment, antigens were first prepared from each of the 14 target microorganisms. In this process, the microorganisms were purchased as frozen glycerol or dried stock of bacteria from the American Type Culture Collection (ATCC). After thawing and reconstitution inside an anaerobic chamber according to standard practices, a loop of each microorganism was separately streaked onto plates, which were incubated at 37° C. under appropriate aerobic or anaerobic culture conditions for one to five days to obtain isolated colonies.

To maintain a stock of isolates at −80° C., an isolated colony was picked from each agar plate and resuspended in 5 ml of pre-reduced thioglycolate broth, then incubated at 37° for 48 hours. This material was transferred to a cryogenic tube, and glycerol was be added at 15%, after which the tube was frozen at −80° C.

To validate the identity of each cultured isolate, one colony from each was picked and suspended in 5 ml thioglycolate broth. A Gram stain was prepared according to standard technique and examined microscopically to determine the Gram-staining characteristics of the isolate. Further confirmation of microorganism identity was performed by use of biochemical tests included on the API Rapid ID system, and comparison with Rapid ID biochemical and metabolic profiles for each species.

To prepare bacterial cell extracts for use as antigens, cultures of each target microorganism were harvested as follows: one colony of each microorganism was picked from the agar plate and suspended in 5 ml thioglycolate broth. This suspension was used to inoculate 20 mL of thioglycolate broth for each anaerobe, and into 20 mL of tryptic soy (TS) broth for each aerobe. Plates and tubes were incubated under appropriate aerobic and anaerobic conditions for up to three days at 37° C. Cultures were harvested by centrifugation and pellets were suspended in phosphate-buffered saline (PBS), washed twice in PBS and frozen at −80° C. until further processing.

To determine the density of microorganisms in each culture, the CFU count was done as follows: 10-fold serial dilutions of the re-suspended colonies from the preceding step were prepared. 100 microliters of the last 3 serial dilutions were spread onto agar plates in triplicate and incubated at 37° C for up to 3 days. Colonies on plates were counted to calculate the bacterial concentration of the isolates in CFU/mL. Concentration of microorganisms in CFU/mL were also determined by optical density measurements at 600 nm.

To prepare antigens for use in inoculating laying hens, the following steps were carried out: Aliquots of harvested bacterial cells (thawed as needed) were adjusted to a concentration of 10\hat{ }6 CFU/mL. Bacterial cells were inactivated by treatment with formalin 1% overnight at 37° C . Inactivated cells were washed three times with sterile PBS, and a check for viable cells was performed by plating an aliquot of treated cells on agar and overnight incubation under appropriate aerobic or anaerobic conditions. Each preparation adjusted to a concentration of 10\hat{ }9 CFU/mL, then stored at 4° C. until ready for immunization.

Remaining aliquots of each live microorganism culture were stored at −80° C. and used in subsequent testing for target-specificity, killing rates, and other measurements.

Once antigens from all targeted microbiome organisms were obtained and stored, immunization of laying hens began as follows.

Thirty-two female Red Sexlink (Barred Rock/Rhode Island Red crossbred) chickens approximately 19 weeks of age were divided into groups of two, for a total of 16 pairs of hens. Animals were housed on an existing poultry farm, leg-banded to permit positive identification of each pair, and kept pairwise in segregated housing with internal laying boxes, and fenced external runs to preclude intermingling of hens and eggs. Animal care was provided by the poultry farmer, who routinely examined all birds for good health status. All birds were kept in the segregated housing with food (custom-mixed balanced ration for laying hens containing corn, beans, and poultry pre mix (minerals, vitamins, trace elements)) and water ad libitum for two weeks for acclimatization and health monitoring. Egg collection began as soon as hens began laying in their individual nesting boxes. All collected eggs were labeled to correspond to the pair of hens that produced them, and cross-checked against a master list. A sample of eggs from each pair of hens was stored for baseline IgY determination, and all other eggs were destroyed.

Each stored and labeled bacterial antigen was then be prepared to a concentration of 10\hat{ }9 CFU/mL and mixed with aluminum hydroxide and magnesium hydroxide (Imject® Alum) as an adjuvant, and stored at 4° C. until time of immunization.

After the two-week acclimatization period, each of 14 pairs of hens was immunized with 1 mL/hen of the assigned bacterial antigen mixed with adjuvant, by injecting 0.5 mL of the mixture into each breast muscle. Booster immunizations were given at 2, 4, 6, and 8 weeks after the initial immunization. The remaining four hens remained unimmunized, but housed, segregated, in the same facility as immunized hens. Eggs from these hens were used to extract non-specific IgY for determination of baseline level of IgYs specific of any of the target antigens, in later steps.

Beginning at 6 weeks after initial immunization, eggs were collected daily from all 16 pairs of laying hens, and labeled indelibly to correspond with the antigen type used, and then stored at 4° C. until purification of IgY and further analysis.

All of the following steps took place for eggs from each pair of microorganism-immunized hens and for eggs from the unimmunized hens. For purification of egg yolk IgY antibodies, a water dilution method as previously described was used. Intact egg yolks were physically separated from whites and rolled on paper towels to remove adherent whites. The yolk membrane was punctured, and yolk allowed to flow into a graduated cylinder. Yolks from multiple eggs from each pair of similarly-immunized hens was pooled before further use. The pooled yolks were mixed gently with 3.5 volumes of cold acidified distilled water (pH 2.5 adjusted with 0.1 M HCl).

After thorough mixing, the pH was adjusted to the range from 5.0 to 5.2, and incubated at 4° C. for 12 hours. The water-soluble fraction (WSF) was obtained by centrifugation at 9,000×g and 4° C. for 20 minutes, with the supernatant comprising the WSF.

IgY was precipitated by adding saturated sodium sulfate solution to the WSF to achieve a final concentration of 20%, and mixed by repeated inversion every 5 minutes for 20 minutes at room temperature. The mixture was then centrifuged at 4000×g for 30 minutes at 4° C. The pellet was resuspended in phosphate-buffered saline (PBS) to a standard volume of 15 mL and dialyzed against 1% NaCl solution. Following dialysis, IgY was frozen at −20° C. in plastic containers.

Total concentration of target-specific IgY in yolks was then determined using an enzyme-linked immunosorbent assay (ELISA). To determine the concentration of target-specific IgY, wells of microliter plates were coated with 100 microliters of a suspension of at least 10\hat{ }9 formalin-inactivated target microorganisms in carbonate-bicarbonate buffer (0.1 M, pH 9.6) and incubated overnight at 4° C. Target-specific IgY from each pair of similarly-immunized hens was diluted by 50% and incubated in microplate wells in PBS-Tween with 2% non-fat milk at 37° C for 1 h. Microplate wells were washed three times with 1×PBS, 0.05% Tween-20. Then, 100 microliters of goat-anti-chicken IgY conjugated to horseradish peroxidase (HR) was added, and washed three times with 1×PBS, 0.05% Tween-20. Finally, HR substrate was added, and plates were scanned using spectrophotometry at 652 nm to detect concentration of target-specific IgY.

IgY quality was assessed by analysis on SDS-PAGE, stained with Coomassie brilliant blue R-250, to assure that bands representing the heavy and light chains of IgY were detected.

During IgY production, eggs from each pair of immunized hens were collected weekly. Eggs were separated and the yolks processed as described above to extract the IgY, and stored, labeled, at −20 C until further testing and use.

Samples from each of the 15 batches of IgY, comprised of 14 batches of target-specific IgY, and 1 batch of non-target-specific IgY extracted from the eggs of unimmunized hens was used in the following steps for analysis.

Growth inhibition assays were conducted to detect the degree of inhibition induced by each of the 14 batches of IgY at various concentrations. Isolates of all target microorganisms were grown in 5 mL thioglycolate broth for anaerobes, and 5 mL TS broth for aerobes, and their turbidity adjusted to 0.05-0.2 optical density (OD) units on a spectrophotometer, consistent with bacterial concentrations of 10\hat{ }6 to 10\hat{ }8 CFU/mL. IgY in PBS was prepared and filter-sterilized through a 0.22 micron filter. 100 microliters of prepared IgY was added to 100 microliters of the appropriate aerobic or anaerobic culture medium, and incubated at 37° C. for up to 3 days, during which time growth was determined by periodic optical density measurements until the end of the experiment.

The above steps were repeated iteratively for all 14 target-specific IgY and for the IgY from unimmunized hens, against all 14 bacterial targets, to determine the growth inhibition of each IgY against each target.

Specific activity for each of the 15 IgY preparations (14 target-specific and one non-target-specific) was determined by Enzyme-Linked Immunosorbent Assay (ELISA). A 96-well microtiter plate was coated overnight at 4° C. in carbonate-bicarbonate buffer with 100 microliter of antigens from targeted bacteria.

Prepared plates were washed thrice with PBS, pH 8.0 and 0.05% Tween 20. 200 microliters of PBS-Tween were added to each well and incubated at 37° C for 60 minutes to block non-specific binding sites in the wells. Each of the 15 IgY preparations was added to the wells of the pre-coated microtiter plate with antigens from targeted and non-targeted bacteria in separate wells and in duplicate, and incubated for 90 minutes at 37° C. The plates were washed thrice with PBS, pH 8.0 and 0.05% Tween 20.

One hundred microliters of goat anti-chicken IgG conjugated with horseradish peroxidase, diluted to 10.000× with PBS-Tween 20, were added to each well and incubated for 90 minutes at 37° C. After three washes with PBS-Tween 20, 100 microliters of substrate solution (TMB Liquid-1 Component) were added to develop a measurable color. Absorbance was then measured at 450 nm using spectrophotometry. This procedure indicated the degree to which each target-specific IgY, as well as the non-target-specific IgY, binds to each of the 14 bacterial antigens targeted by the 14 target-specific IgYs.

Results: PRODUCTION OF TARGET-SPECIFIC IGY: Table 1 shows that total IgY from each pair of immunized hens was elevated in concentration compared with eggs from the unimmunized, control hens. Titers of total IgY in immunized hens reached levels of between 47.12 to 69.81 mg/mL by 8 weeks after initial immunization. Titers of microorganism-specific IgY reached levels of between 21.02 and 35.70 mg/mL of yolk by 8 weeks after initial immunization.

TABLE 1

| | | | | |
|---|---|---|---|---|
| | Total | Total | Specific | Control |
| | Protein | IgY | IgY | IgY |
| Target Microorganism | mg/ml | mg/ml | mg/ml | mg/ml |

DIO MICE TARGETS

| | | | | |
|---|---|---|---|---|
| Bacteroides ovatus | 157.16 | 69.35 | 31.58 | 4.06 |
| Lachnospira sp | 132.18 | 60.89 | 33.64 | 4.14 |
| Clostridiales difficile | 156.98 | 54.73 | 18.51 | 4.52 |
| Lactococcus lactis | 161.64 | 54.73 | 30.88 | 4.62 |
| Bacteroides | 160.69 | 47.12 | 21.02 | 6.73 |
| thetaiotaomicron | | | | |
| Ruminococus sp | 208.98 | 48.64 | 35.70 | 4.42 |

HUMAN IBS TARGETS

| | | | | |
|---|---|---|---|---|
| Bacteroides ovatus | 157.16 | 69.35 | 31.58 | 4.06 |
| Alistipes putriqinis | 114.54 | 65.00 | 9.18 | 5.52 |
| Blautia coccoides | 146.27 | 69.81 | 22.71 | 4.68 |
| Veillonella atypica | 70.67 | 62.56 | 21.07 | 4.39 |
| Ruminococcus torques | 143.31 | 63.93 | 25.79 | 4.40 |
| Escherichia coli | 81.94 | 59.06 | 26.46 | 9.54 |
| Enterococcus faecalis | 76.89 | 33.95 | 9.17 | 8.07 |
| Klebsiella pneumoniae | 107.92 | 63.32 | 10.79 | 6.37 |
| Pseudomonas aeruginosa | 114.51 | 65.37 | 37.51 | 4.36 |
| Non-immunized (Control) | 123.93 | 55.27 | NA | NA |

Table 1 illustrates the results for the experiment described in Example 1, showing successful production of target-specific IgY against each of 14 bacterial targets, six in the group identified as having increased abundance in diet-induced obese (DIO) mice, and nine in the group identified as having increased abundance in human studies of the microbiome in IBS. One microorganism, B. ovatus, is present in both groups, making the total 14.

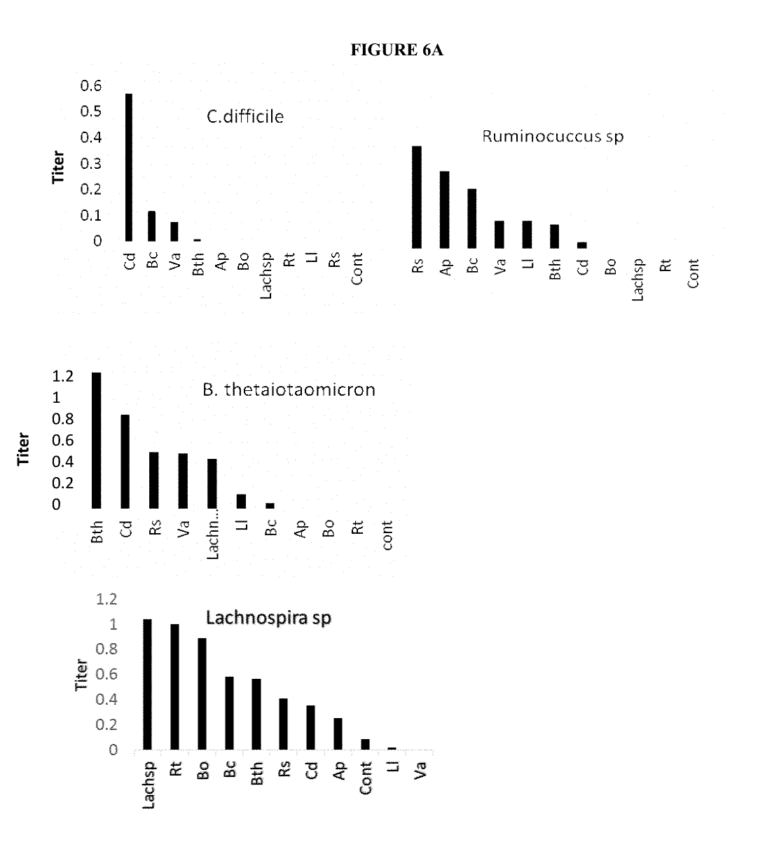

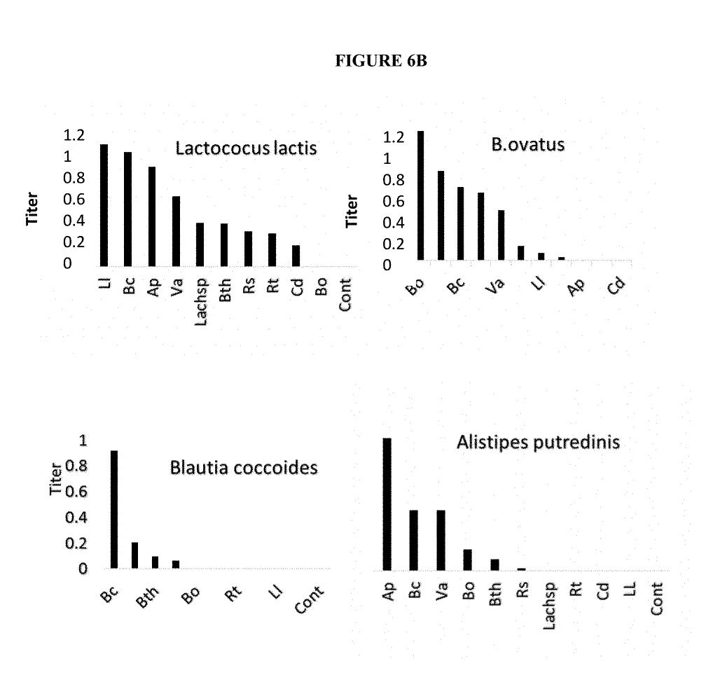

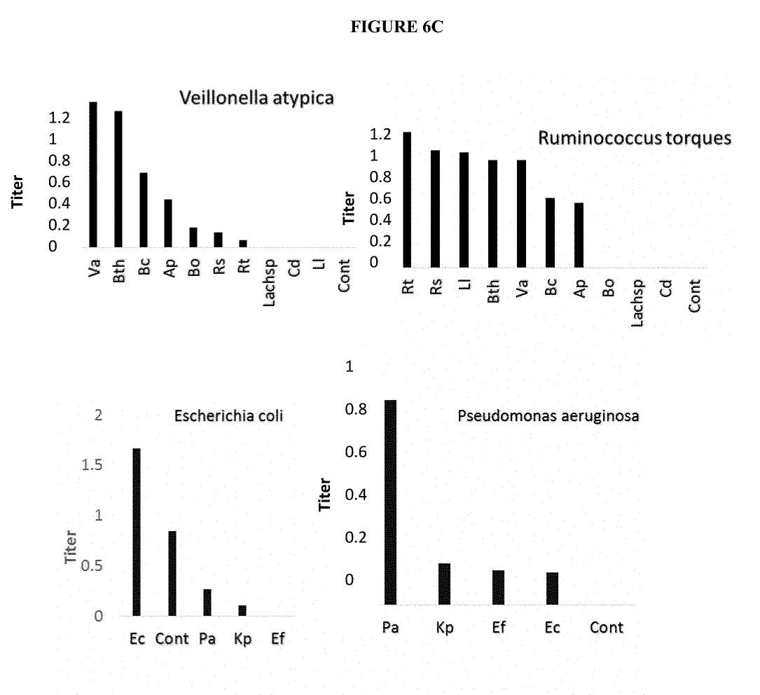

DETERMINATION OF SPECIFIC ACTIVITY: FIGS. 6A, 6H, 6C and 6D provide a series of bar graphs of ELISA results. The TITLE of each graph indicates the name of each target-specific IgY, while the X-AXIS labels indicate all microorganisms in the experiment. Height of the bars indicate binding affinity of the target-specific IgY for each microorganism. These graphs illustrate that a) each target-specific IgY binds most avidly to its cognate microorganism (C. difficile, for example, binds with highest affinity to C. difficile microorganisms), b) that non-target-specific IgY from unimmunized hens (Control) demonstrate little or no detectable activity against most target microorganism antigens, with the exception of K. pneumoniae and E. faecalis, and c) that each target-specific IgY demonstrated strongest activity against its intended target antigens, with lesser activity against antigens from non-targeted microorganisms.

It will be evident to one skilled in the art that the graphs in FIGS. 6A,6B,6C and 6D support the use of target-specific IgY in a future diagnostic embodiment of this invention, in which a specimen from a microbiome will be added to an ELISA comprised of IgY against microorganisms known to be of importance in the relation between a microbiome and a health state, and the quantitative results of said ELISA are used to indicate relative abundance of target microorganisms in said microbiome specimen.

DETERMINATION OF GROWTH INHIBITION OF TARGET MICROORGANISMS: Table 2 illustrates the rates of growth inhibition, compared with untreated cultures, of targeted bacteria in culture by specific IgY and by control IgY extracted from eggs of unimmunized hens. Inspection of the Table reveals significant growth inhibition by specific IgY against all target species except E. faecalis and K. pneumoniae, as would be expected from the poor specificity identified in FIGS. 6A, 6B, 6C and 6D. Inhibition of growth of R. torques and V. atypica tended towards significance.

TABLE 2

| Specific IgY | | Control IgY | |
|---|---|---|---|
| Inhibition Rate | | Inhibition Rate | |
| (vs. untreated) | P- | (vs. untreated) | P- |
| (%) | Value | (%) | Value |

DIO MICE TARGETS

| | | | | |
|---|---|---|---|---|
| Bacteroides ovatus | 65.92 | 0.017 | −5.57 | 0.669 |
| Lachnospira sp | 57.25 | 0.000 | 6.92 | 0.575 |
| Clostridium difficile | 67.84 | 0.000 | 4.06 | 0.931 |
| Lactococcus lactis | 52.97 | 0.002 | −0.32 | 0.979 |
| Bacteroides | 57.77 | 0.021 | 9.02 | 0.555 |
| thetaiotaomicron | | | | |
| Ruminococcus sp | 49.39 | 0.042 | 3.57 | 0.834 |

HUMAN IBS TARGETS

| | | | | |
|---|---|---|---|---|
| Alistipes putridinis | 50.77 | 0.005 | −4.57 | 0.714 |
| Blautia coccoides | 51.45 | 0.038 | 6.87 | 0.670 |
| Veillonella atypica | 52.16 | 0.099 | 13.19 | 0.575 |
| Ruminococcus torques | 51.46 | 0.079 | 10.06 | 0.628 |
| Escherichia coli | 31.62 | 0.021 | 12.19 | 0.028 |
| Enteroeoccus faecalis | 1.37 | 0.699 | −0.40 | 0.945 |
| Klebsiella pneumonia | 9.73 | 0.117 | 17.46 | 0.075 |
| Pseudomonas aeruginosa | 47.07 | 0.009 | 16.13 | 0.051 |

Table 2 shows the rate of growth inhibition of cultures of each targeted microorganism by target-specific IgY and by control IgY from non-immunized hens' eggs in Example 1. The comparison in both cases is against cultures that were not treated with either kind of IgY. Significant inhibition is indicated in the Table by bold print,

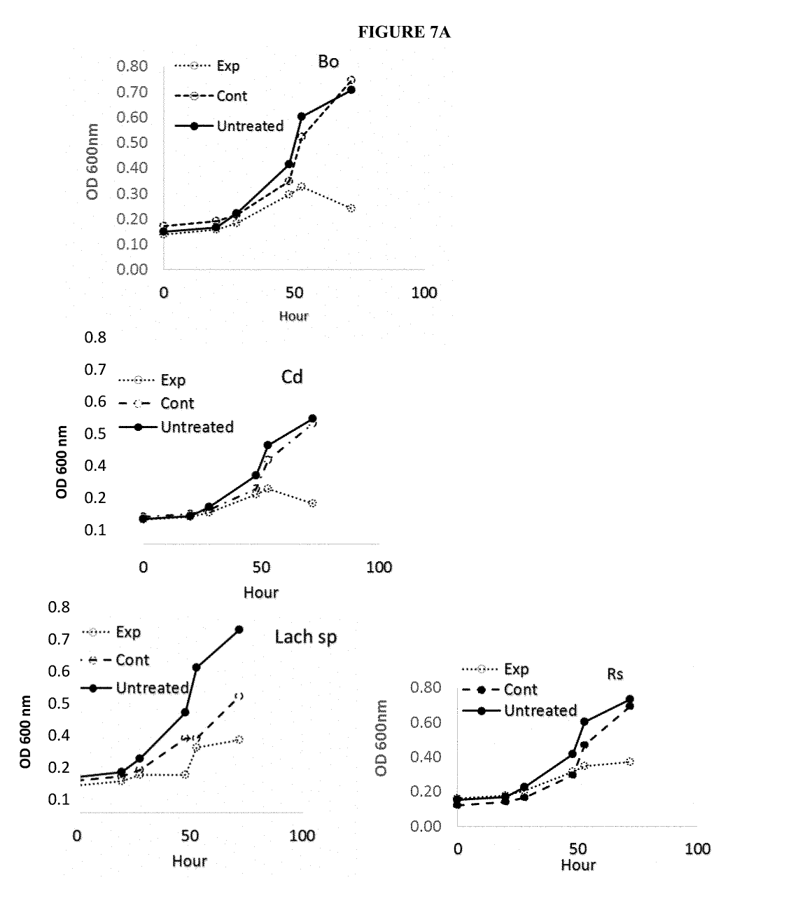

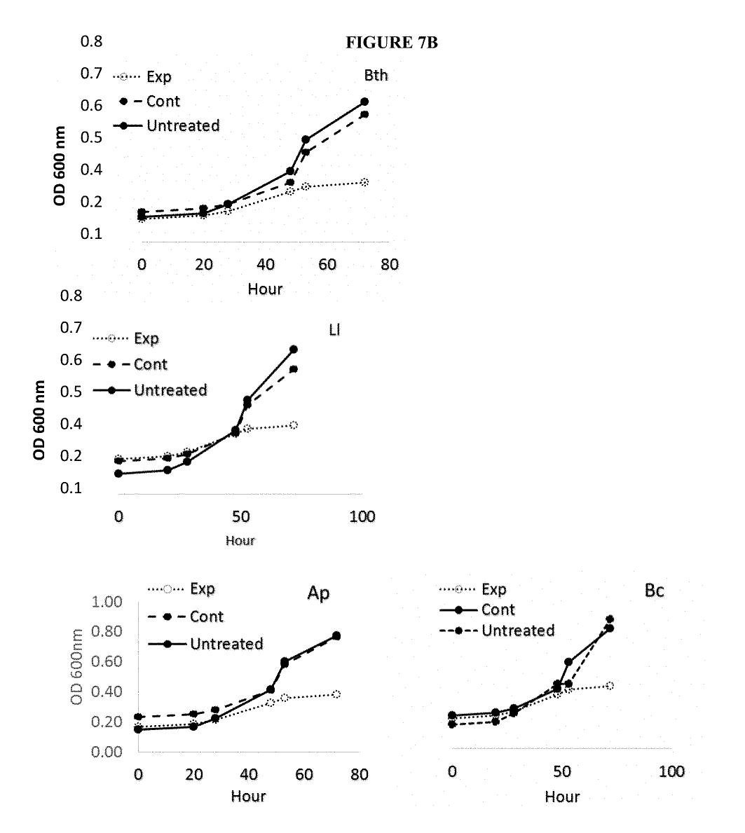

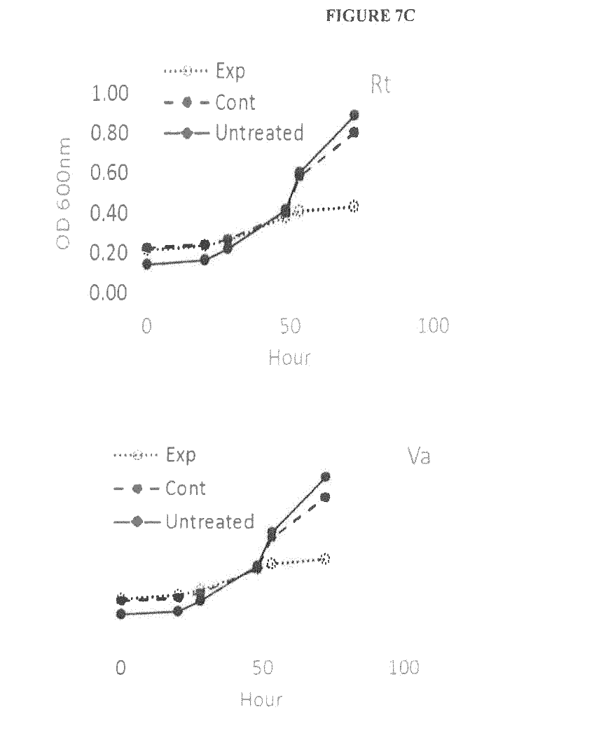

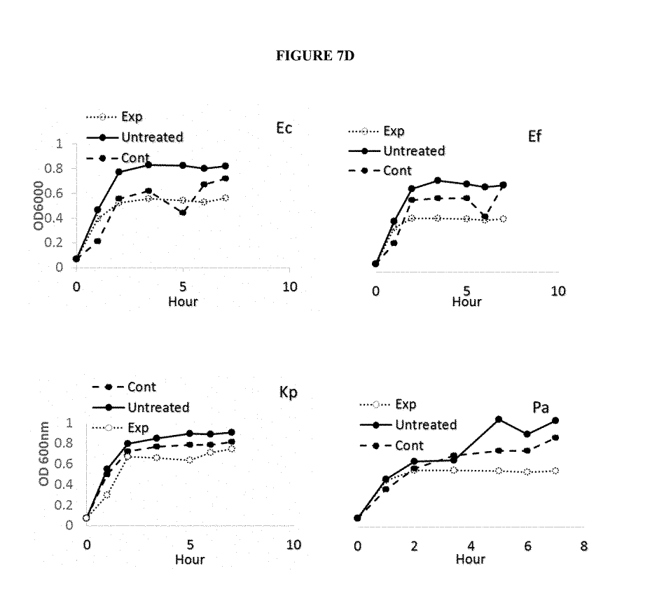

FIGS. 7A, 7B, 7C and 7D are a set of graphs demonstrating the growth inhibition over time for each of the 14 target microorganisms exposed to no treatment (Untreated), control IgY from unimmunized hens' eggs (Cont), and the experimental IgY corresponding to each organism (Exp). These graphs reflect the data found in Table 2.

In summary, this example demonstrated that a) IgY production against individual species in a microbiome is practical, b) the approximate amount of targeted IgY that is produced per egg is 21.02 and 35.70 mg/mL, c) significant in vitro growth inhibition produced by most target-specific IgY against its cognate target, and e) the specificity of activity of each target-specific IgY against its cognate target. This information was then used for formulation of test products for use in mammalian subjects, specifically mice with diet-induced obesity (DIO).

### Example 2

### Demonstration of Impact of Target-Specific IgY Administration on Gut Microbiome Composition in a Living Animal Model

Materials and Methods: The six target-specific IgY compounds prepared in Example 1 from the comparison of microbiome composition between lean and diet-induced obese (DIO) mice were mixed and shipped to a contract research organization (CRO) for administration to experimental DIO mice. As a control solution, an equivalent concentration of IgY extracted from eggs of unimmunized hens was shipped to the CRO for administration to control DIO mice.

The mixed IgY formulation as prepared provided an average of 174.12 mg/day of each of the six individual IgYs. This dose is consistent with published studies of IgY for treating enteric pathogens, in which 100-200 mg/kg/day have been used. Table 3 shows the calculated dose of target-specific IgY for each target organism.

TABLE 3

| | DOSE/DAY | Dose/day |
|---|---|---|
| Target Microorganism | (mg) | (mg/kg) |
| Bacteroides ovatus | 47.36 | 192.53 |
| Lachnospira sp | 50.47 | 205.15 |
| Clostriodes difficile | 27.76 | 112.84 |
| Lactococcus lactis | 46.32 | 188.31 |
| Bacteroides thetaiotaomicron | 31.53 | 128.18 |
| Ruminococus sp | 53.55 | 217.69 |

Average dose: 174.12 mg/kg

Table 3 shows the dose/day, and dose in mg/kg, of each of the six target-specific IgYs used in the DIO mouse experiment described in Example 2.

At the CRO facility, 12 DIO mice underwent acclimatization on a standard high-fat diet and water for 5 days, and a baseline fecal specimen was taken from each individual for a microbiome profile. Six animals were then be fed the customized IgY-containing formulation for 20 days, and six animals were fed a formulation of IgY from non-immunized eggs for the same period. Administration of study or control material was performed by orogastric tube gavage three times daily, administering 0.5 mL of the appropriate formulation at each session. A follow up microbiome profile was performed on each animal on Day 20 of the study.

In addition, blood specimens were obtained from all animals on Day 0 and Day 20 of the study to determine basic metabolic parameters and levels of three markers of inflammation (IL-6, tumor necrosis factor alpha (TNF-alpha), and C-reactive protein (CRP). Finally, baseline and final levels of serum zonulin were measured to detect changes in intestinal permeability.

All study animals were weighed daily from initiation of the study to its termination 20 days later.

Baseline and end-of-study microbiome profiles and biomarkers were compared statistically to identify changes from baseline in relative abundance of the targeted microorganisms within the entire microbiome.

The ratios of each target microorganism's abundance in the experimental to the control groups were determined at baseline and at the end of study. A reduction in at least one of these ratios was considered to demonstrate the target-specific effectiveness of the at least one IgY.

Results: Reductions in the ratios of relative abundance in experimental vs. control microbiomes of three targeted organisms (B. ovatus, B. thetaiotaomicron, and Lachnospiraceae 28-4) were observed at the end of the study. Specifically, the ratio of abundance of Bacteroides ovatus between experimental and control groups was 0.781 at baseline, but 0.612 at completion of the study; the baseline ratio for Bacteroides thetaiotaomicron was 0.820 and at study completion 0.797; for Lachnospiraceae bacterium 28-4 the baseline ratio was 1.021 and final ratio was 0.817. These findings demonstrate the ability of target-specific IgY to reduce a high relative abundance of a targeted organism known to be elevated in a disease process, here, diet-induced obesity.

Table 4 shows that administration of both control IgY from unimmunized hens' eggs and target-specific IgY produced significant changes in both targeted and non-targeted microorganisms over the time of the study. Double underlined rows indicate species targeted by experimental IgY; bold text highlights cells with significant differences between baseline and final species abundance. While 27 significant shifts were demonstrated in the control group, including three of the 6 targeted species, only 12 significant shifts were shown in the experimental group, including the same 3 of 5 targets. This finding indicates that the experimental IgY has greater target-specificity than does the control IgY.

A significantly greater reduction in body weight was demonstrated from baseline to final in the experimental group compared with control animals.

Specifically, control animals weighed an average of 44 grams at baseline, and an average of 41.12 grams at end of study, a loss of 2.88 grams that was not statistically significant, while experimental animals weighed an average of 41.27 grams at baseline and 37.83 grams at end of study, a significant loss of 3.43 grains (p=0.040 for the within-group comparison). These findings demonstrate an alteration in phenotype in the animals treated with a microbiome-altering formulation of target-specific IgY.

No animals in either group had elevated markers of inflammation (TNF-alpha, IL-6, or CRP) at study baseline, and no significant increases were demonstrated between the groups at end of study. No significant alterations in serum zonulin, a measure of intestinal permeability, were demonstrated between baseline and final measures between groups, but zonulin in the experimental group was significantly higher compared to baseline (112.5 ng/mL vs. 96.1 ng/mL), with no significant increase in the control group (99.1 vs. 106.8 ng/mL). These findings indicate no observed impact of IgY treatment on inflammatory markers, and a small but significant increase in intestinal permeability in the treated group, evidence of a small biological impact of treatment of uncertain importance.

In summary, this Example demonstrates that treatment of DIO mice having a dysbiotic microbiome with a formulation of IgY molecules targeting microorganisms found in higher abundance in diseased (DIO) than in healthy (lean) animals does reduce the ratio of said microorganisms in treated vs. untreated animals, indicating the predicted reductive effect on the microbiome. The Example also demonstrates the predicted phenotypic change (significant weight loss over time) in the treated vs. untreated animals.

The disclosures of each and every patent, patent application, and publication cited herein are hereby incorporated herein by reference in their entirety.

It is thought that the method and process of the present invention will be understood from the foregoing description and it will be apparent that various changes may be made in the form, or implementation thereof without departing from the spirit and scope of the invention or sacrificing all of its material advantages, the form hereinbefore described being merely a preferred or exemplary embodiment thereof.

What is claimed:

1. A method for selectively altering the composition of a microbiome in at least one subject in a fashion capable of preventing, reducing the risk of, or treating, a disease associated with characteristics of a microbiome, the method comprising administering to the at least one subject a therapeutically effective amount of at least one immunoglobulin Y (IgY) specific of
a. at least one microorganism in a microbiome, or
b. at least one functional microorganism-produced molecule;
wherein the microbiome is selected from the group consisting of intestinal, gastric, oral, oropharyngeal, otic, nasal, nasopharyngeal, skin, axillary, vaginal, and conjunctival microbiomes; and
wherein the administered therapeutically effective amount of the at least one IgY reduces the relative abundance of at least one microorganism in the microbiome of at least one subject.

2. The method of claim 1, wherein the at least one microorganism is a non-pathogen selected from the group consisting of bacteria, fungi, yeasts, viruses, prions, parasites, and other life forms comprising a microbiome of the subject.

3. The method of claim 1, wherein the at least one microorganism is a pathogen selected from the group consisting of bacteria, fungi, yeasts, viruses, prions, parasites, and other life forms with pathogenic potential in the subject.

4. The method of claim 1, wherein the at least one microorganism comprises at least one strain, taxon or operational taxonomic unit (OTU) that is associated with a specific disease or a disease risk when present on a microbiome profile at a relative abundance higher than a reference value.

5. The method of claim 1, wherein the relative abundance of the at least one microorganism is reduced by about 20% to about 60% as compared with its relative abundance prior to administration of the therapeutically effective amount of at least one IgY.

6. The method of claim 1, wherein the at least one IgY targets at least one strain, species, or other taxonomic unit known to be associated with disease or disease risk when present at high relative abundance in the microbiome of the at least one subject.

7. The method of claim 1, wherein the IgY targets at least one microorganism-produced molecule known to be associated with disease or disease risk when present in the at least one subject.

8. The method of claim 1, wherein the at least one IgY targets at least one microorganism-produced molecule, said at least one microorganism-produced molecule comprising at least one selected from the group including, but not limited to, a lipopolysaccharide (LPS), a flagellin, an adhesion factor, an endotoxin, an exotoxin, a verotoxin, a virulence factor, a bacterioein, or a peripheral membrane protein.

9. The method of claim 1, wherein the at least one subject is monitored for a change in at least one characteristic selected from the group consisting of: a manifestation of a disease or disease risk, a biomarker relevant to a disease or disease risk, a measure of microbiome diversity, a measure of at least one microorganism-produced molecule, and a relative abundance of at least one microorganism as compared with a pre-treatment relative abundance.

10. A method for formulating a mixture of IgYs specific of at least one microorganism or microorganism-produced molecule in a microbiome of at least one subject in need thereof, the method comprising:
a. identifying the at least one microorganism in relative abundance higher than a reference value, or;
b. identifying the at least one microcrganism-produced molecule, and;
c. customizing the mixture of IgYs to target and reduce the relative abundance of the at least one microorganism; or the at least one microorganism-produced molecule.

11. A method for increasing the relative abundance of at least one microorganism in a microbiome of at least one subject in need thereof, the method comprising administering to the at least one subject a therapeutically effective amount of at least one probiotic bacterial species or live biothcrapeutic agent, wherein the need of the at least one subject is determined by detecting a relative abundance of the at least one microorganism lower than a reference value in the microbiome of the at least one subject.

12. The method of claim 11, wherein the at least one live biotherapeutic or probiotic bacterial species is:
a. A non-pathogen found in the microbiome of the at least one subject, or
b. A non-pathogen live biotherapeutic or probiotic species or strain known to increase the relative abundance of microorganisms found in the microbiome of at least one subject, or
c. A non-pathogen live biotherapeutic agent or probiotic species known to ameliorate symptoms, or to normalize biomarkers, associated with the at least one health condition undergoing prevention or treatment.

13. A method for formulating a therapeutically effective mixture of live biotherapeutic or probiotic species specific of at least one microorganism in a microbiome of at least one subject in need thereof, the method comprising identifying at least one microorganism present in a relative abundance lower than a reference value in a microbiome profile of the at least one subject, wherein the mixture of probiotics is customized to target and increase the relative abundance of the at least one microorganism.

14. A method for formulating a therapeutically effective mixture of both target-specific IgY and specific live biotherapeutic or probiotic microorganisms in the microbiome of at least one subject in need thereof, the method comprising identifying at least one microorganism present in a relative abundance higher than a reference value in a microbiome profile, and at least one microorganism present in a relative abundance lower than a reference value in a microbiome profile, wherein the mixture of target-specific IgY and live biotherapeutic or probiotic species is customized to restore the relative abundances of said microorganism towards that of a reference value.

15. A method for formulating mixtures of target-specific IgY into preparations for administration to research animals for the facilitation of research into microbiomes, the method comprising identification of at least one microorganism the relative abundance of which researchers wish to change, or at least one functional microorganism-produced molecule the concentration of which researchers wish to change, in an animal or laboratory in vitro model of a microbiome in order to observe resulting changes in host phenotypic expression, in relative abundance of other microorganisms in the microbiome, in concentration of other microorganism-produced molecules, or in biomarkers associated with microbiome composition.
